(19) United States
(12) Patent Application Publication
(10) Pub. No.: US 2026/0218139 A1
(43) Pub. Date: Jul. 30, 2026

(54) IMPROVED ENZYMES AND METHODS FOR THE SYNTHESIS OF CANNABINOIDS

(71) Applicant: Cellibre, Inc., San Diego, CA (US)

(72) Inventors: Spiros Kambourakis, San Diego, CA (US); Russell Scott Komor, San Diego, CA (US); Nicky Christopher Caiazza, Encinitas, CA (US); Nicholas Donald Keul, San Diego, CA (US); Caleb Marshall Walker, San Diego, CA (US)

(21) Appl. No.: 19/141,576

(22) PCT Filed: Dec. 19, 2023
(86) PCT No.: PCT/US2023/084953

§ 371 Date: Jun. 20, 2025

Related U.S. Application Data
(60) Provisional application No. 63/433,676, filed on Dec. 19, 2022.

(51) Int. Cl. C12N 9/10; C12N 1/16; C12N 1/20; C12N 9/00; C12P 7/42; C12R 1/01; C12R 1/645

(52) U.S. Cl. CPC C12N 9/1085; C12N 1/16; C12N 1/20; C12N 9/93; C12P 7/42; C12R 2001/01; C12R 2001/645; C12Y 205/01; C12Y 602/01002

(57) ABSTRACT

Disclosed herein are novel CBGA and CBGVA synthases and methods for improvement of their overall activities for the synthesis of CBGA and CBGVA from their respective precursors, olivetolic acid (OA) or divarinic acid (DVA) and GPP. Also disclosed are fusion proteins to enhance synthesis of CBGA and CBGVA. The methods described herein also increase the titer and the purity of CBGA and CBGVA made by a cell by 1) decreasing the formation of byproducts FCBGA and FCBGVA that are synthesized from the respective prenylation of OA and DVA with FPP, 2) increasing the intracellular availability of OA and DVA and 3) increasing the speed of CBGA and CBGVA formation, while reducing accumulation of intermediates.

### RELATED APPLICATION

This application claims priority to, and the benefit of, co-pending U.S. Provisional Application No. 63/433,676, filed Dec. 19, 2022. The disclosure of said provisional application is hereby incorporated by reference in its entirety.

### BACKGROUND OF THE INVENTION

The Cannabaceae family of plants produces numerous different cannabinoids (>=120) in variable, relative quantities over a 7-10 week flowering period. Many of these cannabinoids have been and are currently being explored as therapeutics in chordates (e.g., mammals), and as a result, they are largely approved for medical and/or recreational use in the United States (Abrams D I Eur J Int Med 2018, 49, 7-11). Specifically, the most sought after (phyto) cannabinoids are: tetrahydrocannabinolic acid (THCA), cannabidiolic acid (CBDA), and cannabichromenic acid (CBCA). These phytocannabinoids and their associated chemical analogs are all biosynthesized in various quantities from the same pre-cursor: cannabigerolic acid (CBGA). Thus, to mass produce any specific phytocannabinoid (e.g., THCA/CBDA/CBCA/etc), both the rate and total quantity of biosynthesized CBGA must be enhanced and increased (Blatt-Janmaat K, Qu, Y. Int J Mol Sci 2021, 22 (5), 2454). One of the key bottlenecks to increasing CBGA titers is the activity of a prenyl transferase (PT), the enzyme that combines geranyl pyrophosphate (GPP) and olivetolic acid (OA) to form CBGA. Various soluble and membrane bound prenyltransferases have been described that perform this reaction, one of the most successful being MPT4, a membrane bound enzyme from Cannabis. Even this enzyme, however, needs to be improved in order to increase the titers and productivities of CBGA and its derived cannabinoids.

In addition to CBGA, cannabinoids derived from cannabigerovarinic acid (CBGVA) are commercial products with tetrahydrocannabirarinic acid (THCVA) being currently the most popular. Increasing the titer and productivity of CBGVA is therefore critical to creating industrial organisms for the synthesis of these types of cannabinoids (THCVA, CBCVA, CBDVA, etc). The activity of the prenyltransferase that condenses GPP and DVA to form CBGVA is one of the key limitations to increase CBGVA titers. The cannabis prenyltransferase MPT4 has 3-5 times lower activity for prenylating DVA, and as a result a better prenyltransferase for making CBGVA is required.

### SUMMARY OF THE INVENTION

Herein we disclose both soluble and membrane bound prenyltransferases with increased activity for the synthesis of CBGA and CBVA. Other modifications of the host strain that increase availability of GPP and DVA from butyryl-CoA have been achieved in our labs and are described in co-owned PCT Application No. PCT/US2022/046926, incorporated by reference herein in its entirety. Described herein are novel CBGA and cannabigerovarinic acid (CBGVA) synthases and methods for improvement of their overall activities for the synthesis of CBGA and CBGVA from their respective precursors, olivetolic acid (OA) or divarinic acid (DVA) and geranyl pyrophosphate (GPP). In some embodiments, methods described herein also increase the titer and the purity of CBGA and CBGVA made by a cell by i) decreasing the formation of byproducts farnesyl cannabigerolic acid (FCBGA) and farnesyl cannabigerovarinic acid (FCBGVA) that are synthesized from the respective prenylation of OA and DVA with farnesyl pyrophosphate (FPP) (e.g., FIG. 1), and ii) increasing the speed of CBGA and CBGVA formation, while reducing accumulation of intermediates. In addition to providing mutant membrane bound prenyltransferases (MPT) and soluble aromatic prenyltransferases (APT) which show improved selectivity for GPP as well as enhanced production of CBGA and CBVA, provision of fusion proteins which contain a prenyltransferase and a GPP synthase, resulting in an overall increase in CBGA production. Thus, the present invention pertains, in some embodiments, to mutant prenyltransferases, mutant prenyltransferases that are binary fusion proteins between GPP synthase and a prenyltransferase (soluble or membrane bound), to effectuate an increased titer and purity of CBGA and CBGVA.

Some aspects of the present disclosure are directed to an aromatic prenyltransferase (APT) engineered to transfer geranyl pyrophosphate (GPP) to olivetolic acid and/or divarinic acid with an at least two times higher efficiency compared to a mutant aromatic prenyltransferase APT73.77 (SEQ ID NO: 38), wherein said engineered APT contains an amino acid sequence with at least two amino acid modifications compared to SEQ ID NO: 38, wherein a first of the at least two amino acid modifications corresponds to a substitution, deletion or insertion at amino acid position R162 of SEQ ID NO: 38.

In some embodiments, a second of the at least two amino acid modifications compared to the amino acid sequence APT73.77 is selected from a substitution, deletion or insertion at an amino acid position corresponding to position H39, V41, Q127, E130, V131, 1156, R162, N164, R205, Δ223, H225, Q227, G254, G260, L276, G280, R282, C283, L284, D285, or G286 of SEQ ID NO: 38.

In some embodiments, the at least two amino acid modifications comprise a second, third, fourth, and fifth or more amino acid modifications, selected from a substitution, deletion or insertion at four or more amino acid positions corresponding to positions H39, V41, Q127, E130, V131, 1156, R162, N164, R205, Δ223, H225, Q227, G254, G260, L276, G280, R282, C283, L284, D285, and/or G286 of SEQ ID NO: 38, wherein the modifications are not the substitution R205S.

In some embodiments, the engineered APT comprises an amino acid sequence with at least 90% identity to the amino acid sequence of APT73.179 (SEQ ID NO: 1), APT73.119 (SEQ ID NO: 2), APT73.159 (SEQ ID NO: 3), APT73.160 (SEQ ID NO: 4), APT73.161 (SEQ ID NO: 5), APT73.162 (SEQ ID NO: 6), APT73.163 (SEQ ID NO: 7), APT73.165 (SEQ ID NO: 8), APT73.166 (SEQ ID NO: 9), APT73.168 (SEQ ID NO: 10), APT73.169 (SEQ ID NO: 11), APT73.170 (SEQ ID NO: 12), APT73.172 (SEQ ID NO: 13), APT73.182 (SEQ ID NO: 14), NO: 17), APT73.186 (SEQ ID NO: 18), APT73.187 (SEQ ID NO: 19), APT73.188 (SEQ ID NO: 20), APT73.189 (SEQ ID NO:21), APT73.190 (SEQ ID NO: 22), APT73.191 (SEQ ID NO: 23), APT73.192 (SEQ ID NO: 24), APT73.193 (SEQ ID NO: 25), APT73.194 (SEQ ID NO: 26), APT73.195 (SEQ ID NO: 27), APT73.196 (SEQ ID NO: 28), APT73.197 (SEQ ID NO: 29), APT73.199 (SEQ ID NO: 31), APT73.200 (SEQ ID NO: 32), APT73.201 (SEQ ID NO: 33), APT73.202 (SEQ ID NO: 34), APT73.203 (SEQ ID NO: 35), APT73.204 (SEQ ID NO: 36), APT73.248 (SEQ ID NO: 96) or a functional fragment or variant thereof. In some embodiments, the engineered APT comprises an amino acid sequence with at least 90% identity to the amino acid sequence of APT73.187 (SEQ ID NO: 96) or APT73.248 (SEQ ID NO: 96).

In some embodiments, the at least two amino acid modifications comprise, three or more amino acid modifications comprising the substitution R162Q, one or more substitutions selected from the group consisting of Q127E, E130R, V131I, and Δ280G, and one or more substitutions selected from the group consisting of H39V, V41C, and V41L, all as relating to the amino acid sequence of APT73.77 (SEQ ID NO: 38).

In some embodiments, the engineered APT comprises an amino acid sequence with at least one amino acid modification as compared to APT73.179 (SEQ ID NO: 1), APT73.119 (SEQ ID NO: 2), APT73.159 (SEQ ID NO: 3), APT73.160 (SEQ ID NO: 4), APT73.161 (SEQ ID NO: 5), APT73.162 (SEQ ID NO: 6), APT73.163 (SEQ ID NO: 7), APT73.165 (SEQ ID NO: 8), APT73.166 (SEQ ID NO: 9), APT73.168 (SEQ ID NO: 10), APT73.169 (SEQ ID NO: 11), APT73.170 (SEQ ID NO: 12), APT73.172 (SEQ ID NO: 13), APT73.182 (SEQ ID NO: 14), APT73.183 (SEQ ID NO: 15), APT73.184 (SEQ ID NO: 16), APT73.185 (SEQ ID NO: 17), APT73.186 (SEQ ID NO: 18), APT73.187 (SEQ ID NO: 19), APT73.188 (SEQ ID NO: 20), APT73.189 (SEQ ID NO: 21), APT73.190 (SEQ ID NO: 22), APT73.191 (SEQ ID NO: 23), APT73.192 (SEQ ID NO: 24), APT73.193 (SEQ ID NO: 25), APT73.194 (SEQ ID NO: 26), APT73.195 (SEQ ID NO: 27), APT73.196 (SEQ ID NO: 28), APT73.197 (SEQ ID NO: 29), APT73.199 (SEQ ID NO: 31), APT73.200 (SEQ ID NO: 32), APT73.201 (SEQ ID NO: 33), APT73.202 (SEQ ID NO: 34), APT73.203 (SEQ ID NO: 35), APT73.204 (SEQ ID NO: 36), APT73.248 (SEQ ID NO: 96), or a functional fragment or variant thereof.

In other aspects, the disclosure is directed to a recombinant membrane-bound prenyltransferase (rMPT), said rMPT engineered to transfer geranyl pyrophosphate (GPP) to olivetolic acid and/or divarinic acid with higher efficiency compared to a naturally occurring MPT, wherein said rMPT has an amino acid sequence comprising at least one amino acid modification compared to the amino acid sequence of MPT4.1 (SEQ ID NO: 46), MPT48 (SEQ ID NO: 47), or MPT69 (SEQ ID NO: 48).

In some embodiments, said rMPT has at least one amino acid modification at a position corresponding to amino acid positions 29, 72, 135, and/or 254 of SEQ ID NO: 46 (MPT4.1). In some embodiments, the at least one amino acid modification compared to SEQ ID NO: 46 (MPT4.1) is selected from at least one of M29I, M29V, I72C, I72V, I72A, Δ135M and/or V254L.

In some embodiments, the rMPT comprises an amino acid sequence with at least 90% identity to the amino acid sequence of MPT4.33 (SEQ ID NO: 39), MPT4.29 (SEQ ID NO: 40), MPT4.30 (SEQ ID NO: 41), MPT4.32 (SEQ ID NO: 42), MPT4.34 (SEQ ID NO: 43), MPT4.40 (SEQ ID NO: 44), MPT4.41 (SEQ ID NO: 45).

In some embodiments, the rMPT comprises an amino acid sequence having at least one amino acid modification as compared to SEQ ID NO: 47 (MPT48) producing a mutant with one or both of i) increased activity for prenylation of OA and DVA to form CBGA and CBGVA, respectively, and ii) increased selectivity for GPP over FPP.

In some embodiments, the at least one amino acid modification compared to MPT48 (SEQ ID NO: 47) comprises one or more deletion, insertion or substitution one or more amino acid positions corresponding to the first 50 amino acids of the N-terminus M1-G50 and/or H54, M88, R89, C93, Δ94, N96, D97, V98, V99, D100, Q101, D102, F103, D104, R109, R113, S121, Δ136, C147, Q148, V154, K165, Q172, L175, T178, L179, 1181, L201, T207, V214, Y217, D218, V219, Y221, T253, L254, T262, V267, N269, P271, L281, Δ284, Δ287, S304, G305, W306, N314, L316, G317, G318, V327, M329, and/or L330 of SEQ ID NO: 47.

In some embodiments, the rMPT comprises an amino acid sequence having at least one amino acid modification as compared to SEQ ID NO: 48 (MPT69) producing a mutant with one or both of i) increased activity for prenylation of OA and DVA to form CBGA and CBGVA, respectively, and ii) increased selectivity for GPP over FPP.

In some embodiments, the at least one amino acid modification as compared to MPT69 (SEQ ID NO: 48) comprises one or more deletion, insertion or substitution at one or more amino acid positions corresponding to the first 45 amino acids of the N-terminus M1-G45, and/or H49, M83, R84, C88, Δ89, N91, D92, N93, 194, D95, Q96, D97, F98, D99, R104, R108, S116, Δ131, C142, H143, V149, K160, Q167, L170, T173, L174, Δ176, R196, T202, V209, Y212, D213, V214, Y216, T248, C249, V257, V262, N264, P266, L276, Δ279, Δ282, S299, G300, W301, N309, M311, S312, G313, Δ322, L324, and/or L325 of SEQ ID NO: 48.

In some embodiments, the rMPT comprises an amino acid sequence with at least 90% identity to the amino acid sequence of MPT69.2 (SEQ ID NO: 98), MPT69.5 (SEQ ID NO: 99), MPT69.6 (SEQ ID NO: 100), MPT69.7 (SEQ ID NO: 101), MPT69.8 (SEQ ID NO: 102), MPT69.9 (SEQ ID NO: 103), or MPT69.10 (SEQ ID NO: 104).

In some aspects, the disclosure is directed to a cell comprising an APT and/or a rMPT as described herein, wherein the cell is capable of producing CBGA in the presence of GPP and OA. In some embodiments, the cell is capable of producing CBGVA in the presence of GPP and DVA. In some embodiments, the cell is capable of making a cannabinoid in the presence of a carbon source and, optionally, hexanoic or butyric acid.

In some embodiments, the cell expresses an exogenous membrane transporter that improves OA or DVA uptake. In some embodiments, one or more genes in the cell encoding a protein that exports OA or DVA is down-regulated or deactivated. In some embodiments, the cell encodes an exogenous hexanoyl-CoA synthetase and/or butyryl-CoA synthetase.

In some embodiments, the cell is a yeast cell or a bacterial cell. In some embodiments, the yeast cell is a Yarrowia strain or a Saccharomyces strain.

In other aspects, the disclosure is directed to a method of producing CBGA, CBGVA or derivatives thereof comprising culturing a cell as described herein under suitable conditions to produce CBGA, CBGVA or derivatives thereof. In some embodiments, the suitable condition comprises supplementing a culture media in which the cell is cultured with at least one of butyric acid, valeric acid, isovaleric acid, hexanoic acid, hexanol, butanol, oleic acid, glycerol or glucose.

Some aspects of the disclosure are directed to a fusion protein comprising a polypeptide having geranyl diphosphate synthase (GPS) activity fused directly or indirectly to a polypeptide having prenyltransferase activity, wherein the polypeptide having prenyltransferase activity comprises a) a polypeptide sequence having at least 85% identity to the polypeptide sequence of MPT48 (SEQ ID NO: 47), or MPT69 (SEQ ID NO: 48), b) a polypeptide sequence comprising at least one amino acid modification compared to the amino acid sequence of MPT4.1 (SEQ ID NO: 46), or c) a polypeptide comprising i) an amino acid sequence with at least two amino acid modifications compared to SEQ ID NO: 38, wherein a first of the at least two amino acid modifications corresponds to a substitution, deletion or insertion at amino acid position R162 of SEQ ID NO: 38, and wherein the N-terminal of the polypeptide having prenyltransferase activity is fused to the C-terminal of the GPS, or the C-terminal of the polypeptide having prenyltransferase activity is fused to the N-terminal of the GPS.

In some embodiments, the fusion protein comprises a polypeptide sequence having at least 90% identity to GPS1.1-L18-APT73.119 (SEQ ID NO: 52), GPS1.1-L18-APT73.159 (SEQ ID NO: 53), GPS1.1-L18-APT73.160 (SEQ ID NO: 54), GPS1.1-L18-APT73.161 (SEQ ID NO: 55), GPS1.1-L18-APT73.162 (SEQ ID NO: 56), GPS1.1-L18-APT73.163 (SEQ ID NO: 57), GPS1.1-L18-APT73.165 (SEQ ID NO: 58), GPS1.1-L18-APT73.166 (SEQ ID NO: 59), GPS1.1-L18-APT73.168 (SEQ ID NO: 60), GPS1.1-L18-APT73.169 (SEQ ID NO: 61), GPS1.1-L18-APT73.170 (SEQ ID NO: 62), GPS1.1-L18-APT73.172 (SEQ ID NO: 63), GPS1.1-L18-APT73.179 (SEQ ID NO: 64), GPS1.1-L18-APT73.182 (SEQ ID NO: 65), GPS1.1-L18-APT73.183 (SEQ ID NO: 66), GPS1.1-L18-APT73.184 (SEQ ID NO: 67), GPS1.1-L18-APT73.185 (SEQ ID NO: 68), GPS1.1-L18-APT73.186 (SEQ ID NO: 69), GPS1.1-L18-APT73.187 (SEQ ID NO: 70), GPS1.1-L18-APT73.188 (SEQ ID NO: 71), GPS1.1-L18-APT73.189 (SEQ ID NO: 72), GPS1.1-L18-APT73.190 (SEQ ID NO: 73), GPS1.1-L18-APT73.191 (SEQ ID NO: 74), GPS1.1-L18-APT73.192 (SEQ ID NO: 75), GPS1.1-L18-APT73.193 (SEQ ID NO: 76), GPS1.1-L18-APT73.194 (SEQ ID NO: 77), GPS1.1-L18-APT73.195 (SEQ ID NO: 78), GPS1.1-L18-APT73.196 (SEQ ID NO: 79), GPS1.1-L18-APT73.197 (SEQ ID NO: 80), GPS1.1-L18-APT73.199 (SEQ ID NO: 82), GPS1.1-L18-APT73.200 (SEQ ID NO: 83), GPS1.1-L18-APT73.201 (SEQ ID NO: 84), GPS1.1-L18-APT73.202 (SEQ ID NO: 85), GPS1.1-L18-APT73.203 (SEQ ID NO: 86), GPS1.1-L18-APT73.204 (SEQ ID NO: 87), or GPS1.1-L18-APT73.248 (SEQ ID NO: 97).

In some embodiments, the fusion protein comprises a polypeptide sequence having at least 90% identity to GPS1.1-L11-MPT4.1 (SEQ ID NO: 88), GPS1.1-L11-MPT4.29 (SEQ ID NO: 89), GPS1.1-L11-MPT4.30 (SEQ ID NO: 90), GPS1.1-L11-MPT4.32 (SEQ ID NO: 91), GPS1.1-L11-MPT4.33 (SEQ ID NO: 92), GPS1.1-L11-MPT4.34 (SEQ ID NO: 93), GPS1.1-L11-MPT4.40 (SEQ ID NO: 94), GPS1.1-L11-MPT4.41 (SEQ ID NO: 95).

In some embodiments, the polypeptide having prenyltransferase activity has improved selectivity for GPP over FPP, as compared to a control membrane bound prenyltransferase or soluble aromatic prenyltransferase. In some embodiments, the polypeptide having Geranyl diphosphate synthase activity comprises a polypeptide of SEQ ID NO: 49 (GPS1.1), or a functional fragment or functional variant thereof.

In some embodiments, the fusion protein further comprises a linker polypeptide between the polypeptide having Geranyl diphosphate synthase activity and the polypeptide having prenyltransferase activity. In some embodiments, the linker comprises a polypeptide selected from the amino acid sequence of SEQ ID NO: 50 or SEQ ID NO: 51.

In some embodiments, the fusion protein comprises the polypeptide sequence of GPS1.1-L18-APT73.119 (SEQ ID NO: 52), GPS1.1-L18-APT73.159 (SEQ ID NO: 53), GPS1.1-L18-APT73.160 (SEQ ID NO: 54), GPS1.1-L18-APT73.161 (SEQ ID NO: 55), GPS1.1-L18-APT73.162 (SEQ ID NO: 56), GPS1.1-L18-APT73.163 (SEQ ID NO: 57), GPS1.1-L18-APT73.165 (SEQ ID NO: 58), GPS1.1-L18-APT73.166 (SEQ ID NO: 59), GPS1.1-L18-APT73.168 (SEQ ID NO: 60), GPS1.1-L18-APT73.169 (SEQ ID NO: 61), GPS1.1-L18-APT73.170 (SEQ ID NO: 62), GPS1.1-L18-APT73.172 (SEQ ID NO: 63), GPS1.1-L18-APT73.179 (SEQ ID NO: 64), GPS1.1-L18-APT73.182 (SEQ ID NO: 65), GPS1.1-L18-APT73.183 (SEQ ID NO: 66), GPS1.1-L18-APT73.184 (SEQ ID NO: 67), GPS1.1-L18-APT73.185 (SEQ ID NO: 68), GPS1.1-L18-APT73.186 (SEQ ID NO: 69), GPS1.1-L18-APT73.187 (SEQ ID NO: 70), GPS1.1-L18-APT73.188 (SEQ ID NO: 71), GPS1.1-L18-APT73.189 (SEQ ID NO: 72), GPS1.1-L18-APT73.190 (SEQ ID NO: 73), GPS1.1-L18-APT73.191 (SEQ ID NO: 74), GPS1.1-L18-APT73.192 (SEQ ID NO: 75), GPS1.1-L18-APT73.193 (SEQ ID NO: 76), GPS1.1-L18-APT73.194 (SEQ ID NO: 77), GPS1.1-L18-APT73.195 (SEQ ID NO: 78), GPS1.1-L18-APT73.196 (SEQ ID NO: 79), GPS1.1-L18-APT73.197 (SEQ ID NO: 80), GPS1.1-L18-APT73.199 (SEQ ID NO: 82), GPS1.1-L18-APT73.200 (SEQ ID NO: 83), GPS1.1-L18-APT73.201 (SEQ ID NO: 84), GPS1.1-L18-APT73.202 (SEQ ID NO: 85), GPS1.1-L18-APT73.203 (SEQ ID NO: 86), GPS1.1-L18-APT73.204 (SEQ ID NO: 87), or GPS1.1-L18-APT73.248 (SEQ ID NO: 97), or a functional fragment or variant thereof.

In some embodiments, the fusion protein comprises the polypeptide sequence of GPS1.1-L11-MPT4.1 (SEQ ID NO: 88), GPS1.1-L11-MPT4.29 (SEQ ID NO: 89), GPS1.1-L11-MPT4.30 (SEQ ID NO: 90), GPS1.1-L11-MPT4.32 (SEQ ID NO: 91), GPS1.1-L11-MPT4.33 (SEQ ID NO: 92), GPS1.1-L11-MPT4.34 (SEQ ID NO: 93), GPS1.1-L11-MPT4.40 (SEQ ID NO: 94), GPS1.1-L11-MPT4.41 (SEQ ID NO: 95), or a functional fragment or variant thereof.

In another aspect, the disclosure is directed to a cell comprising the fusion protein as described herein, wherein the cell is capable of producing CBGA in the presence of OA and GPP. In some embodiments, the cell is capable of producing CBGVA in the presence of DVA and a GPP. In some embodiments, the cell is capable of making a cannabinoid in the presence of a carbon source and, optionally, hexanoic or butyric acid. In some embodiments, the cell expresses an exogenous membrane transporter that improves OA or DVA uptake. In some embodiments, one or more genes in the cell encoding a protein that exports OA or DVA is down-regulated or deactivated.

In some embodiments, the cell encodes an exogenous hexanoyl-CoA synthetase and/or butyryl-CoA synthetase. In some embodiments, the cell is a yeast cell or a bacterial cell. In some embodiments, the yeast cell is a Yarrowia strain or a Saccharomyces strain.

In another aspect, the disclosure is directed to a method of producing CBGA, CBGVA or derivatives thereof comprising culturing a cell as described herein under suitable conditions to produce CBGA, CBGVA or derivatives thereof. In some embodiments, suitable condition comprises supplementing a culture media in which the cell is cultured with at least one of butyric acid, valeric acid, isovaleric acid, hexanoic acid, hexanol, butanol, oleic acid, glycerol or glucose.

### BRIEF DESCRIPTION OF THE DRAWINGS

Embodiments of the invention are further described by way of the following FIGURE.

FIG. 1A depict CBGA derivatives synthesized by CBGA synthase(s) described herein.

FIG. 1B depict FCBGA derivatives synthesized by CBGA synthase(s) described herein.

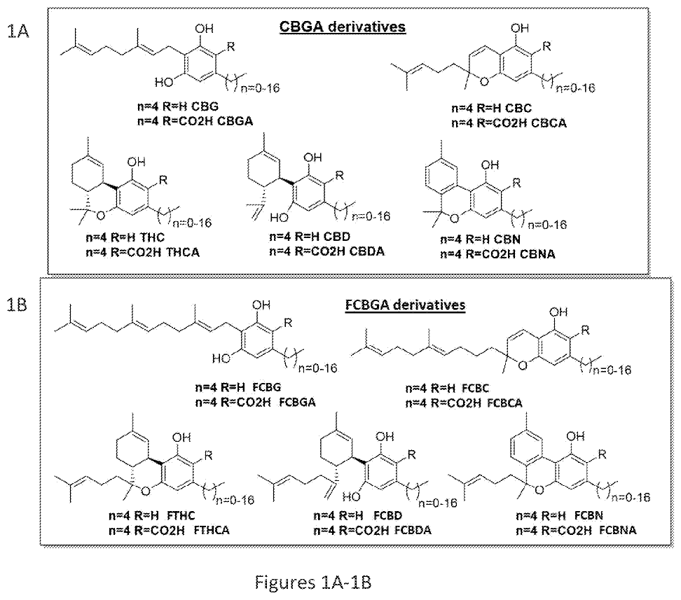

### DETAILED DESCRIPTION OF THE INVENTION

Some aspects of the present disclosure are directed to an aromatic prenyltransferase (APT) engineered to transfer geranyl pyrophosphate (GPP) to olivetolic acid and/or divarinic acid with an at least two times higher efficiency compared to a mutant aromatic prenyltransferase APT73.77 (SEQ ID NO: 38), wherein said engineered APT contains an amino acid sequence with at least two amino acid modifications compared to SEQ ID NO: 38, wherein a first of the at least two amino acid modifications corresponds to a substitution, deletion or insertion at amino acid position R162 of SEQ ID NO: 38.

Other aspects of the present disclosure are directed to an aromatic prenyltransferase (APT) engineered to transfer geranyl pyrophosphate (GPP) to olivetolic acid and/or divarinic acid with an at least twenty times higher efficiency compared to a naturally occurring aromatic prenyltransferase APT73 (SEQ ID NO: 37), wherein said engineered APT contains an amino acid sequence with at least two amino acid modifications compared to SEQ ID NO: 37, wherein a first of the at least two amino acid modifications corresponds to a substitution, deletion or insertion at amino acid position R162 of SEQ ID NO: 37.

Other aspects of the present invention are directed to an aromatic prenyltransferase (APT), said APT engineered to transfer geranyl pyrophosphate (GPP) to olivetolic acid and/or divarinic acid with an equal or higher efficiency compared to a mutant aromatic prenyltranferase APT73.119 (SEQ ID NO: 2), wherein the engineered APT comprises an amino acid sequence with at least 90% identity to the amino acid sequence of SEQ ID NO 2, and wherein at least four of the amino acids of the engineered APT corresponding to amino acids at positions 41, 127, 130, 131, 162 and 280 of SEQ ID NO 2 are identical.

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 1 (APT73.179). In some embodiments, the APT comprises an amino acid sequence with at least 90% identity to SEQ ID NO: 1 (APT73.179). In some embodiments, the APT comprises a functional fragment of SEQ ID NO: 1 (APT73.179) or an amino acid sequence with at least 70%, 75%, 80%, 85%, 90%, 95%, 96%, 97%, 98%, 98.5%, 99%, 99.5%, 99.9%, or 100% identity to SEQ ID NO: 1 (APT73.179). In some embodiments, the functional fragment of SEQ ID NO: 1 (APT73.179) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 2 (APT73.119). In some embodiments, the APT comprises an amino acid sequence with at least 90% identity to SEQ ID NO: 2 (APT73.119). In some embodiments, the APT comprises a functional fragment of SEQ ID NO: 2 (APT73.119) or an amino acid sequence with at least 70%, 75%, 80%, 85%, 90%, 95%, 96%, 97%, 98%, 98.5%, 99%, 99.5%, 99.9%, or 100% identity to SEQ ID NO: 2 (APT73.119). In some embodiments, the functional fragment of SEQ ID NO: 2 (APT73.119) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 3 (APT73.159). In some embodiments, the APT comprises an amino acid sequence with at least 90% identity to SEQ ID NO: 3 (APT73.159). In some embodiments, the APT comprises a functional fragment of SEQ ID NO: 3 (APT73.159) or an amino acid sequence with at least 70%, 75%, 80%, 85%, 90%, 95%, 96%, 97%, 98%, 98.5%, 99%, 99.5%, 99.9%, or 100% identity to SEQ ID NO: 3 (APT73.159). In some embodiments, the functional fragment of SEQ ID NO: 3 (APT73.159) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 4 (APT73.160). In some embodiments, the APT comprises an amino acid sequence with at least 90% identity to SEQ ID NO: 4 (APT73.160). In some embodiments, the APT comprises a functional fragment of SEQ ID NO: 4 (APT73.160) or an amino acid sequence with at least 70%, 75%, 80%, 85%, 90%, 95%, 96%, 97%, 98%, 98.5%, 99%, 99.5%, 99.9%, or 100% identity to SEQ ID NO: 4 (APT73.160). In some embodiments, the functional fragment of SEQ ID NO: 4 (APT73.160) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 5 (APT73.161). In some embodiments, the APT comprises an amino acid sequence with at least 90% identity to SEQ ID NO: 5 (APT73.161). In some embodiments, the APT comprises a functional fragment of SEQ ID NO: 5 (APT73.161) or an amino acid sequence with at least 70%, 75%, 80%, 85%, 90%, 95%, 96%, 97%, 98%, 98.5%, 99%, 99.5%, 99.9%, or 100% identity to SEQ ID NO: 5 (APT73.161). In some embodiments, the functional fragment of SEQ ID NO: 5 (APT73.161) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, 99.9%, or 100% identity to SEQ ID NO: 6 (APT73.162). In some embodiments, the APT comprises an amino acid sequence with at least 90% identity to SEQ ID NO: 6 (APT73.162). In some embodiments, the APT comprises a functional fragment of SEQ ID NO: 6 (APT73.162) or an amino acid sequence with at least 70%, 75%, 80%, 85%, 90%, 95%, 96%, 97%, 98%, 98.5%, 99%, 99.5%, 99.9%, or 100% identity to SEQ ID NO: 6 (APT73.162). In some embodiments, the functional fragment of SEQ ID NO: 6 (APT73.162) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 7 (APT73.163). In some embodiments, the APT comprises an amino acid sequence with at least 90% identity to SEQ ID NO:
7 (APT73.163). In some embodiments, the APT comprises a functional fragment of SEQ ID NO: 7 (APT73.163) or an amino acid sequence with at least 70%, 75%, 80%, 85%, 90%, 96%, 97%, 98%, 98.5%, 95%, 99%, 99.5%, 99.9%, or 100% identity to SEQ ID NO: 7 (APT73.163). In some embodiments, the functional fragment of SEQ ID NO: 7 (APT73.163) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 8 (APT73.165). In some embodiments, the APT comprises an amino acid sequence with at least 90% identity to SEQ ID NO: 8 (APT73.165). In some embodiments, the APT comprises a functional fragment of SEQ ID NO: 8 (APT73.165) or an amino acid sequence with at least 70%, 75%, 80%, 85%, 90%, 95%, 96%, 97%, 98%, 98.5%, 99%, 99.5%, 99.9%, or 100% identity to SEQ ID NO: 8 (APT73.165). In some embodiments, the functional fragment of SEQ ID NO: 8 (APT73.165) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 9 (APT73.166). In some embodiments, the APT comprises an amino acid sequence with at least 90% identity to SEQ ID NO: 9 (APT73.166). In some embodiments, the APT comprises a functional fragment of SEQ ID NO: 9 (APT73.166) or an amino acid sequence with at least 70%, 75%, 80%, 85%, 90%, 95%, 96%, 97%, 98%, 98.5%, 99%, 99.5%, 99.9%, or 100% identity to SEQ ID NO: 9 (APT73.166). In some embodiments, the functional fragment of SEQ ID NO: 9 (APT73.166) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 10 (APT73.168). In some embodiments, the APT comprises an amino acid sequence with at least 90% identity to SEQ ID NO: 10 (APT73.168). In some embodiments, the APT comprises a functional fragment of SEQ ID NO: 10 (APT73.168) or an amino acid sequence with at least 70%, 75%, 80%, 85%, 90%, 95%, 96%, 97%, 98%, 98.5%, 99%, 99.5%, 99.9%, or 100% identity to SEQ ID NO: 10 (APT73.168). In some embodiments, the functional fragment of SEQ ID NO: 10 (APT73.168) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 11 (APT73.169). In some embodiments, the APT comprises an amino acid sequence with at least 90% identity to SEQ ID NO: 11 (APT73.169). In some embodiments, the APT comprises a functional fragment of SEQ ID NO: 11 (APT73.169) or an amino acid sequence with at least 70%, 75%, 80%, 85%, 90%, 95%, 96%, 97%, 98%, 98.5%, 99%, 99.5%, 99.9%, or 100% identity to SEQ ID NO: 11 (APT73.169). In some embodiments, the functional fragment of SEQ ID NO: 11 (APT73.169) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 12 (APT73.170). In some embodiments, the APT comprises an amino acid sequence with at least 90% identity to SEQ ID NO: 12 (APT73.170). In some embodiments, the APT comprises a functional fragment of SEQ ID NO: 12 (APT73.170) or an amino acid sequence with at least 70%, 75%, 80%, 85%, 90%, 95%, 96%, 97%, 98%, 98.5%, 99%, 99.5%, 99.9%, or 100% identity to SEQ ID NO: 12 (APT73.170). In some embodiments, the functional fragment of SEQ ID NO: 12 (APT73.170) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 13 (APT73.172). In some embodiments, the APT comprises an amino acid sequence with at least 90% identity to SEQ ID NO: 13 (APT73.172). In some embodiments, the APT comprises a functional fragment of SEQ ID NO: 13 (APT73.172) or an amino acid sequence with at least 70%, 75%, 80%, 85%, 90%, 95%, 96%, 97%, 98%, 98.5%, 99%, 99.5%, 99.9%, or 100% identity to SEQ ID NO: 13 (APT73.172). In some embodiments, the functional fragment of SEQ ID NO: 13 (APT73.172) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 14 (APT73.182). In some embodiments, the APT comprises an amino acid sequence with at least 90% identity to SEQ ID NO: 14 (APT73.182). In some embodiments, the APT comprises a functional fragment of SEQ ID NO: 14 (APT73.182) or an amino acid sequence with at least 70%, 75%, 80%, 85%, 90%, 95%, 96%, 97%, 98%, 98.5%, 99%, 99.5%, 99.9%, or 100% identity to SEQ ID NO: 14 (APT73.182). In some embodiments, the functional fragment of SEQ ID NO: 14 (APT73.182) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 15 (APT73.183). In some embodiments, the APT comprises an amino acid sequence with at least 90% identity to SEQ ID NO: 15 (APT73.183). In some embodiments, the APT comprises a functional fragment of SEQ ID NO: 15 (APT73.183) or an amino acid sequence with at least 70%, 75%, 80%, 85%, 90%, 95%, 96%, 97%, 98%, 98.5%, 99%, 99.5%, 99.9%, or 100% identity to SEQ ID NO: 15 (APT73.183). In some embodiments, the functional fragment of SEQ ID NO: 15 (APT73.183) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 16 (APT73.184). In some embodiments, the APT comprises an amino acid sequence with at least 90% identity to SEQ ID NO: 16 (APT73.184). In some embodiments, the APT comprises a functional fragment of SEQ ID NO: 16 (APT73.184) or an amino acid sequence with at least 70%, 75%, 80%, 85%, 90%, 95%, 96%, 97%, 98%, 98.5%, 99%, 99.5%, 99.9%, or 100% identity to SEQ ID NO: 16 (APT73.184). In some embodiments, the functional fragment of SEQ ID NO: 16 (APT73.184) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 17 (APT73.185). In some embodiments, the APT comprises an amino acid sequence with at least 90% identity to SEQ ID NO: 17 (APT73.185). In some embodiments, the APT comprises a functional fragment of SEQ ID NO: 17 (APT73.185) or an amino acid sequence with at least 70%, 75%, 80%, 85%, 90%, 95%, 96%, 97%, 98%, 98.5%, 99%, 99.5%, 99.9%, or 100% identity to SEQ ID NO: 17 (APT73.185). In some embodiments, the functional fragment of SEQ ID NO: 17 (APT73.185) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 18 (APT73.186). In some embodiments, the APT comprises an amino acid sequence with at least 90% identity to SEQ ID NO: 18 (APT73.186). In some embodiments, the APT comprises a functional fragment of SEQ ID NO: 18 (APT73.186) or an amino acid sequence with at least 70%, 75%, 80%, 85%, 90%, 95%, 96%, 97%, 98%, 98.5%, 99%, 99.5%, 99.9%, or 100% identity to SEQ ID NO: 18 (APT73.186). In some embodiments, the functional fragment of SEQ ID NO: 18 (APT73.186) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 19 (APT73.187). In some embodiments, the APT comprises an amino acid sequence with at least 90% identity to SEQ ID NO: 19 (APT73.187). In some embodiments, the APT comprises a functional fragment of SEQ ID NO: 19 (APT73.187) or an amino acid sequence with at least 70%, 75%, 80%, 85%, 90%, 95%, 96%, 97%, 98%, 98.5%, 99%, 99.5%, 99.9%, or 100% identity to SEQ ID NO: 19 (APT73.187). In some embodiments, the functional fragment of SEQ ID NO: 19 (APT73.187) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 20 (APT73.188). In some embodiments, the APT comprises an amino acid sequence with at least 90% identity to SEQ ID NO: 20 (APT73.188). In some embodiments, the APT comprises a functional fragment of SEQ ID NO: 20 (APT73.188) or an amino acid sequence with at least 70%, 75%, 80%, 85%, 90%, 95%, 96%, 97%, 98%, 98.5%, 99%, 99.5%, 99.9%, or 100% identity to SEQ ID NO: 20 (APT73.188). In some embodiments, the functional fragment of SEQ ID NO: 20 (APT73.188) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 21 (APT73.189). In some embodiments, the APT comprises an amino acid sequence with at least 90% identity to SEQ ID NO: 21 (APT73.189). In some embodiments, the APT comprises a functional fragment of SEQ ID NO: 21 (APT73.189) or an amino acid sequence with at least 70%, 75%, 80%, 85%, 90%, 95%, 96%, 97%, 98%, 98.5%, 99%, 99.5%, 99.9%, or 100% identity to SEQ ID NO: 21 (APT73.189). In some embodiments, the functional fragment of SEQ ID NO: 21 (APT73.189) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 22 (APT73.190). In some embodiments, the APT comprises an amino acid sequence with at least 90% identity to SEQ ID NO: 22 (APT73.190). In some embodiments, the APT comprises a functional fragment of SEQ ID NO: 22 (APT73.190) or an amino acid sequence with at least 70%, 75%, 80%, 85%, 90%, 95%, 96%, 97%, 98%, 98.5%, 99%, 99.5%, 99.9%, or 100% identity to SEQ ID NO: 22 (APT73.190). In some embodiments, the functional fragment of SEQ ID NO: 22 (APT73.190) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 23 (APT73.191). In some embodiments, the APT comprises an amino acid sequence with at least 90% identity to SEQ ID NO: 23 (APT73.191). In some embodiments, the APT comprises a functional fragment of SEQ ID NO: 23 (APT73.191) or an amino acid sequence with at least 70%, 75%, 80%, 85%, 90%, 95%, 96%, 97%, 98%, 98.5%, 99%, 99.5%, 99.9%, or 100% identity to SEQ ID NO: 23 (APT73.191). In some embodiments, the functional fragment of SEQ ID NO: 23 (APT73.191) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 24 (APT73.192). In some embodiments, the APT comprises an amino acid sequence with at least 90% identity to SEQ ID NO: 24 (APT73.192). In some embodiments, the APT comprises a functional fragment of SEQ ID NO: 24 (APT73.192) or an amino acid sequence with at least 70%, 75%, 80%, 85%, 90%, 95%, 96%, 97%, 98%, 98.5%, 99%, 99.5%, 99.9%, or 100% identity to SEQ ID NO: 24 (APT73.192). In some embodiments, the functional fragment of SEQ ID NO: 24 (APT73.192) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 25 (APT73.193). In some embodiments, the APT comprises an amino acid sequence with at least 90% identity to SEQ ID NO: 25 (APT73.193). In some embodiments, the APT comprises a functional fragment of SEQ ID NO: 25 (APT73.193) or an amino acid sequence with at least 70%, 75%, 80%, 85%, 90%, 95%, 96%, 97%, 98%, 98.5%, 99%, 99.5%, 99.9%, or 100% identity to SEQ ID NO: 25 (APT73.193). In some embodiments, the functional fragment of SEQ ID NO: 25 (APT73.193) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 26 (APT73.194). In some embodiments, the APT comprises an amino acid sequence with at least 90% identity to SEQ ID NO: 26 (APT73.194). In some embodiments, the APT comprises a functional fragment of SEQ ID NO: 26 (APT73.194) or an amino acid sequence with at least 70%, 75%, 80%, 85%, 90%, 95%, 96%, 97%, 98%, 98.5%, 99%, 99.5%, 99.9%, or 100% identity to SEQ ID NO: 26 (APT73.194). In some embodiments, the functional fragment of SEQ ID NO: 26 (APT73.194) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 27 (APT73.195). In some embodiments, the APT comprises an amino acid sequence with at least 90% identity to SEQ ID NO: 27 (APT73.195). In some embodiments, the APT comprises a functional fragment of SEQ ID NO: 27 (APT73.195) or an amino acid sequence with at least 70%, 75%, 80%, 85%, 90%, 95%, 96%, 97%, 98%, 98.5%, 99%, 99.5%, 99.9%, or 100% identity to SEQ ID NO: 27 (APT73.195). In some embodiments, the functional fragment of SEQ ID NO: 27 (APT73.195) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 28 (APT73.196). In some embodiments, the APT comprises an amino acid sequence with at least 90% identity to SEQ ID NO: 28 (APT73.196). In some embodiments, the APT comprises a functional fragment of SEQ ID NO: 28 (APT73.196) or an amino acid sequence with at least 70%, 75%, 80%, 85%, 90%, 95%, 96%, 97%, 98%, 98.5%, 99%, 99.5%, 99.9%, or 100% identity to SEQ ID NO: 28 (APT73.196). In some embodiments, the functional fragment of SEQ ID NO: 28 (APT73.196) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 29 (APT73.197). In some embodiments, the APT comprises an amino acid sequence with at least 90% identity to SEQ ID NO: 29 (APT73.197). In some embodiments, the APT comprises a functional fragment of SEQ ID NO: 29 (APT73.197) or an amino acid sequence with at least 70%, 75%, 80%, 85%, 90%, 95%, 96%, 97%, 98%, 98.5%, 99%, 99.5%, 99.9%, or 100% identity to SEQ ID NO: 29 (APT73.197). In some embodiments, the functional fragment of SEQ ID NO: 29 (APT73.197) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 30 (APT73.198). In some embodiments, the APT comprises an amino acid sequence with at least 90% identity to SEQ ID NO: 30 (APT73.198). In some embodiments, the APT comprises a functional fragment of SEQ ID NO: 30 (APT73.198) or an amino acid sequence with at least 70%, 75%, 80%, 85%, 90%, 95%, 96%, 97%, 98%, 98.5%, 99%, 99.5%, 99.9%, or 100% identity to SEQ ID NO: 30 (APT73.198). In some embodiments, the functional fragment of SEQ ID NO: 30 (APT73.198) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 31 (APT73.199). In some embodiments, the APT comprises an amino acid sequence with at least 90% identity to SEQ ID NO: 31 (APT73.199). In some embodiments, the APT comprises a functional fragment of SEQ ID NO: 31 (APT73.199) or an amino acid sequence with at least 70%, 75%, 80%, 85%, 90%, 95%, 96%, 97%, 98%, 98.5%, 99%, 99.5%, 99.9%, or 100% identity to SEQ ID NO: 31 (APT73.199). In some embodiments, the functional fragment of SEQ ID NO: 31 (APT73.199) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 32 (APT73.200). In some embodiments, the APT comprises an amino acid sequence with at least 90% identity to SEQ ID NO: 32 (APT73.200). In some embodiments, the APT comprises a functional fragment of SEQ ID NO: 32 (APT73.200) or an amino acid sequence with at least 70%, 75%, 80%, 85%, 90%, 95%, 96%, 97%, 98%, 98.5%, 99%, 99.5%, 99.9%, or 100% identity to SEQ ID NO: 32 (APT73.200). In some embodiments, the functional fragment of SEQ ID NO: 32 (APT73.200) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 33 (APT73.201). In some embodiments, the APT comprises an amino acid sequence with at least 90% identity to SEQ ID NO: 33 (APT73.201). In some embodiments, the APT comprises a functional fragment of SEQ ID NO: 33 (APT73.201) or an amino acid sequence with at least 70%, 75%, 80%, 85%, 90%, 95%, 96%, 97%, 98%, 98.5%, 99%, 99.5%, 99.9%, or 100% identity to SEQ ID NO: 33 (APT73.201). In some embodiments, the functional fragment of SEQ ID NO: 33 (APT73.201) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 34 (APT73.202). In some embodiments, the APT comprises an amino acid sequence with at least 90% identity to SEQ ID NO: 34 (APT73.202). In some embodiments, the APT comprises a functional fragment of SEQ ID NO: 34 (APT73.202) or an amino acid sequence with at least 70%, 75%, 80%, 85%, 90%, 95%, 96%, 97%, 98%, 98.5%, 99%, 99.5%, 99.9%, or 100% identity to SEQ ID NO: 34 (APT73.202). In some embodiments, the functional fragment of SEQ ID NO: 34 (APT73.202) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 35 (APT73.203). In some embodiments, the APT comprises an amino acid sequence with at least 90% identity to SEQ ID NO: 35 (APT73.203). In some embodiments, the APT comprises a functional fragment of SEQ ID NO: 35 (APT73.203) or an amino acid sequence with at least 70%, 75%, 80%, 85%, 90%, 95%, 96%, 97%, 98%, 98.5%, 99%, 99.5%, 99.9%, or 100% identity to SEQ ID NO: 35 (APT73.203). In some embodiments, the functional fragment of SEQ ID NO: 35 (APT73.203) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 36 (APT73.204). In some embodiments, the APT comprises an amino acid sequence with at least 90% identity to SEQ ID NO: 36 (APT73.204). In some embodiments, the APT comprises a functional fragment of SEQ ID NO: 36 (APT73.204) or an amino acid sequence with at least 70%, 75%, 80%, 85%, 90%, 95%, 96%, 97%, 98%, 98.5%, 99%, 99.5%, 99.9%, or 100% identity to SEQ ID NO: 36 (APT73.204). In some embodiments, the functional fragment of SEQ ID NO: 36 (APT73.204) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 96%, 97%, 98%, 98.5%, 99%, 99.5%, or 99.9%, or 100% identity to SEQ ID NO: 96 (APT73.248). In some embodiments, the APT comprises an amino acid sequence with at least 90% identity to SEQ ID NO: 96 (APT73.248). In some embodiments, the APT comprises a functional fragment of SEQ ID NO: 96 (APT73.248) or an amino acid sequence with at least 70%, 75%, 80%, 85%, 90%, 95%, 96%, 97%, 98%, 98.5%, 99%, 99.5%, 99.9%, or 100% identity to SEQ ID NO: 96 (APT73.248). In some embodiments, the functional fragment of SEQ ID NO: 96 (APT73.248) has at least 1, 2, 3, 4, 5, 6, 7, 8, 9, 10, 11, 12, 13, 14, 15, 16, 17, 18, 19, or 20 amino acids added to the carboxy terminus.

In some embodiments, the engineered APT comprises an amino acid sequence containing at least three amino acid modifications at three or more positions corresponding to any three or more amino acids of APT73.77 (SEQ ID NO: 38). In some embodiments, the engineered APT comprises an amino acid sequence containing at least four amino acid modifications at four or more positions corresponding to any four or more amino acids of APT73.77 (SEQ ID NO: 38). In some embodiments, the engineered APT comprises an amino acid sequence containing at least five amino acid modifications at five or more positions corresponding to any five or more amino acids of APT73.77 (SEQ ID NO: 38). In some embodiments, the engineered APT comprises an amino acid sequence containing at least six amino acid modifications at six or more positions corresponding to any six or more amino acids of APT73.77 (SEQ ID NO: 38). In some embodiments, the engineered APT comprises an amino acid sequence containing at least seven amino acid modifications at seven or more positions corresponding to any seven or more amino acids of APT73.77 (SEQ ID NO: 38). In some embodiments, the engineered APT comprises an amino acid sequence containing at least eight amino acid modifications at eight or more positions corresponding to any eight or more amino acids of APT73.77 (SEQ ID NO: 38). In some embodiments, the engineered APT comprises an amino acid sequence containing at least nine amino acid modifications at nine or more positions corresponding to any nine or more amino acids of APT73.77 (SEQ ID NO: 38). In some embodiments, the engineered APT comprises an amino acid sequence containing at least ten amino acid modifications at ten or more positions corresponding to any ten or more amino acids of APT73.77 (SEQ ID NO: 38).

In some embodiments, the engineered APT comprises an amino acid sequence containing at least two, three, four, five, six or more amino acid modifications at two, three, four, five, six or more positions corresponding to any two, three, four, five, six or more amino acids of the naturally occurring APT. In some embodiments, the engineered APT comprises an amino acid sequence containing at 5, 6, 7, 8, 9, 10, 11, 12, 13, 14, 15, 16, 17, 18, 19, 20, 21, 22, 23, 24, 25, 26, 27, 28, 29, 30 or more amino acid modifications compared to the amino acid sequence of a naturally occurring APT. In a preferred embodiment, the engineered APT comprises an amino acid sequence containing 20, 21, 22, 23, 24, 25, 26, 27, 28, 29, 30 or more amino acid modifications compared to the amino acid sequence of a naturally occurring APT. In a most preferred embodiment, the engineered APT comprises an amino acid sequence containing 27, 28, 29, 30, 31, 32 or 33 amino acid modifications compared to the amino acid sequence of a naturally occurring APT. In some embodiments, the amino acid modifications are in comparison to the naturally occurring APT73 (SEQ ID NO: 37). In some embodiments, the engineered APT comprises a 3-45 amino acid truncation at the c-terminus as compared to the naturally occurring APT.

In some embodiments, the engineered APT comprises at least three amino acid modifications, the modifications comprising the substitution R162Q, one or more substitutions selected from the group consisting of Q127E, E130R, and V131I, and one or more substitutions selected from the group consisting of H39V, V41C, V41L and Δ280G, all as relating to the amino acid sequence of mutant APT73.77 (SEQ ID NO: 38) or naturally occurring APT73 (SEQ ID NO: 37). In some embodiments, the engineered APT comprises the substitutions R162R and three or more of Δ280G, Q127E, E130R, V131I, H39V, V41C, and/or V41L. In some embodiments, the engineered APT comprises four or more of the substitutions Δ280G, Q127E, E130R, V131I, R162Q, H39V, V41C, and/or V41L. In some embodiments, the engineered APT comprises five or more of the substitutions Δ280G, Q127E, E130R, V131I, H39V, V41C, R162Q, and/or V41L. In some embodiments, the engineered APT comprises all six of the substitutions Δ280G, Q127E, E130R, R162Q, V131I, H39V and one of substitutions V41C or V41L. In some further embodiments, the amino acid sequence comprises the addition of R/H/Y287, R/Y288, R/G289 as compared to the amino acid sequence of SEQ ID NO: 38.

In some embodiments, the engineered APT does not comprise a substitution selected from I156A, R205S, Δ223S, H225K, G260A, L276Y, R282G, C283A, L284S, and D285N. In some embodiments, the engineered APT does not comprise the substitution R205S. In some embodiments, the engineered APT does not comprise the substitution I156A. In some embodiments, the engineered APT does not comprise the substitution Δ223S. In some embodiments, the engineered APT does not comprise the substitution H225K. In some embodiments, the engineered APT does not comprise the substitution G260A. In some embodiments, the engineered APT does not comprise the substitution L276Y. In some embodiments, the engineered APT does not comprise the substitution R282G. In some embodiments, the engineered APT does not comprise the substitution C283A. In some embodiments, the engineered APT does not comprise the substitution L284S. In some embodiments, the engineered APT does not comprise the substitution D285N.

In some embodiments, the engineered APT does not comprise two or more substitutions selected from I156A, R205S, Δ223S, H225K, G260A, L276Y, R282G, C283A, L284S, and D285N.

In some embodiments, the engineered APT comprises an amino acid sequence containing at least two amino acid modifications at two positions of APT73.119. In some embodiments, the engineered APT comprises an amino acid sequence containing at least three amino acid modifications at three positions of APT73.119. In some embodiments, the engineered APT comprises an amino acid sequence containing at least four amino acid modifications at four positions corresponding to any four of APT73.119. In some embodiments, the engineered APT comprises an amino acid sequence containing at 5, 6, 7, 8, 9, 10, 11, 12, 13, 14, 15, 16, 17, 18, 19, 20, 21, 22, 23, 24, 25, 26, 27, 28, 29, 30 or more amino acid modifications compared to the amino acid sequence of APT73.119. In a preferred embodiment, the engineered APT comprises an amino acid sequence containing 20, 21, 22, 23, 24, 25, 26, 27, 28, 29, 30 or more amino acid modifications compared to APT73.119. In a most preferred embodiment, the engineered APT comprises an amino acid sequence containing 27, 28, 29, 30, 31, 32 or 33 amino acid modifications compared to the amino acid sequence of APT73.119. In some embodiments, the engineered APT comprising amino acid modifications in one or more amino acid positions corresponding to one or more of C41, E127, R130, 1156, Q162, N164, R205, Δ223, H225, Q227, G254, G260, L276, G280, C283, R282, C283, L284, D285, or G286 of SEQ ID NO: 2. In another embodiment, the amino acid sequence of the engineered APT comprises the addition of at least 3 amino acids to the C-terminus compared to the amino acid sequence of APT73.119. In some embodiments, the amino acid sequence comprises the addition of R/H/Y287, R/Y288, R/G289 as compared to the amino acid sequence of SEQ ID NO: 2.

In some embodiments, the APT comprises an amino acid sequence with at least one amino acid modification as compared to APT73.179 (SEQ ID NO: 1), APT73.119 (SEQ ID NO: 2), APT73.159 (SEQ ID NO: 3), APT73.160 (SEQ ID NO: 4), APT73.161 (SEQ ID NO: 5), APT73.162 (SEQ ID NO: 6), APT73.163 (SEQ ID NO: 7), APT73.165 (SEQ ID NO: 8), APT73.166 (SEQ ID NO: 9), APT73.168 (SEQ ID NO: 10), APT73.169 (SEQ ID NO: 11), APT73.170 (SEQ ID NO: 12), APT73.172 (SEQ ID NO: 13), APT73.182 (SEQ ID NO: 14), APT73.183 (SEQ ID NO: 15), APT73.184 (SEQ ID NO: 16), APT73.185 (SEQ ID NO: 17), APT73.186 (SEQ ID NO: 18), APT73.187 (SEQ ID NO: 19), APT73.187 (SEQ ID NO: 19), APT73.188 (SEQ ID NO: 20), APT73.189 (SEQ ID NO: 21), APT73.190 (SEQ ID NO: 22), APT73.191 (SEQ ID NO: 23), APT73.192 (SEQ ID NO: 24), APT73.193 (SEQ ID NO: 25), APT73.194 (SEQ ID NO: 26), APT73.195 (SEQ ID NO: 27), APT73.196 (SEQ ID NO: 28), APT73.197 (SEQ ID NO: 29), APT73.198 (SEQ ID NO: 30), APT73.199 (SEQ ID NO: 31), APT73.200 (SEQ ID NO: 32), APT73.201 (SEQ ID NO: 33), APT73.202 (SEQ ID NO: 34), APT73.203 (SEQ ID NO: 35), APT73.204 (SEQ ID NO: 36), or APT73.248 (SEQ ID NO: 96) or a functional fragment or variant thereof. In some embodiments, the at least one amino acid modification comprises at least two, at least three, at least four, at least five or at least six amino acid modifications.

In other aspects, the disclosure is directed to a recombinant membrane-bound prenyltransferase (rMPT), said rMPT engineered to transfer geranyl pyrophosphate (GPP) to olivetolic acid and/or divarinic acid with higher efficiency compared to a naturally occurring MPT, wherein said rMPT has an amino acid sequence comprising at least one amino acid modification compared to the amino acid sequence of MPT4.1 (SEQ ID NO: 46), MPT48 (SEQ ID NO: 47), or MPT69 (SEQ ID NO: 48). In some embodiments, the at least one amino acid modification comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid modifications compared to the amino acid sequence of MPT4.1 (SEQ ID NO: 46), MPT48 (SEQ ID NO: 47), or MPT69 (SEQ ID NO: 48).

In some embodiments, the at least one amino acid modification compared to MPT48 (SEQ ID NO: 47) comprises a deletion, insertion or substitution at one or more amino acid positions corresponding to the first 50 amino acids of the N-terminus M1-G50 and/or H54, M88, R89, C93, Δ94, N96, D97, V98, V99, D100, Q101, D102, F103, D104, R109, R113, S121, Δ136, C147, Q148, V154, K165, Q172, L175, T178, L179, 1181, L201, T207, V214, Y217, D218, V219, Y221, T253, L254, T262, V267, N269, P271, L281, Δ284, Δ287, S304, G305, W306, N314, L316, G317, G318, V327, M329, and/or L330 of SEQ ID NO: 47. In some embodiments, the rMPT comprises at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine or more of the amino acid modifications. In some embodiments, the rMPT further comprises a truncation (e.g., 1-10 amino acids) at the C and/or N terminus.

In some embodiments, the at least one amino acid modification as compared to MPT69 (SEQ ID NO: 48) comprises a deletion, insertion or substitution at one or more amino acid positions corresponding to the first 45 amino acids of the N-terminus M1-G45, and/or H49, M83, R84, C88, Δ89, N91, D92, N93, 194, D95, Q96, D97, F98, D99, R104, R108, S116, Δ131, C142, H143, V149, K160, Q167, L170, T173, L174, Δ176, R196, T202, V209, Y212, D213, V214, Y216, T248, C249, V257, V262, N264, P266, L276, Δ279, Δ282, S299, G300, W301, N309, M311, S312, G313, Δ322, L324, and/or L325 of SEQ ID NO: 48. In some embodiments, the rMPT comprises at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine or more of the amino acid modifications. In some embodiments, the rMPT further comprises a truncation (e.g., 1-10 amino acids) at the C and/or N terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 98 (MPT69.2). In some embodiments, the rMPT comprises an amino acid sequence with at least 90% identity to SEQ ID NO: 98 (MPT69.2). In some embodiments, the rMPT comprises a functional fragment of SEQ ID NO: 98 (MPT69.2) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 98 (MPT69.2). In some embodiments, the functional fragment of SEQ ID NO: 98 (MPT69.2) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 98 (MPT69.2) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 99 (MPT69.5). In some embodiments, the rMPT comprises an amino acid sequence with at least 90% identity to SEQ ID NO: 99 (MPT69.5). In some embodiments, the rMPT comprises a functional fragment of SEQ ID NO: 99 (MPT69.5) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 99 (MPT69.5). In some embodiments, the functional fragment of SEQ ID NO: 99 (MPT69.5) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 99 (MPT69.5) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 100 (MPT69.6). In some embodiments, the rMPT comprises an amino acid sequence with at least 90% identity to SEQ ID NO: 100 (MPT69.6). In some embodiments, the rMPT comprises a functional fragment of SEQ ID NO: 100 (MPT69.6) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 100 (MPT69.6). In some embodiments, the functional fragment of SEQ ID NO: 100 (MPT69.6) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 100 (MPT69.6) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 101 (MPT69.7). In some embodiments, the rMPT comprises an amino acid sequence with at least 90% identity to SEQ ID NO: 101 (MPT69.7). In some embodiments, the rMPT comprises a functional fragment of SEQ ID NO: 101 (MPT69.7) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 101 (MPT69.7). In some embodiments, the functional fragment of SEQ ID NO: 101 (MPT69.7) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 101 (MPT69.7) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 102 (MPT69.8). In some embodiments, the rMPT comprises an amino acid sequence with at least 90% identity to SEQ ID NO: 102 (MPT69.8). In some embodiments, the rMPT comprises a functional fragment of SEQ ID NO: 102 (MPT69.8) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 102 (MPT69.8). In some embodiments, the functional fragment of SEQ ID NO: 102 (MPT69.8) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 102 (MPT69.8) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 103 (MPT69.9). In some embodiments, the rMPT comprises an amino acid sequence with at least 90% identity to SEQ ID NO: 103 (MPT69.9). In some embodiments, the rMPT comprises a functional fragment of SEQ ID NO: 103 (MPT69.9) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 103 (MPT69.9). In some embodiments, the functional fragment of SEQ ID NO: 103 (MPT69.9) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 103 (MPT69.9) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 104 (MPT69.10). In some embodiments, the rMPT comprises an amino acid sequence with at least 90% identity to SEQ ID NO: 104 (MPT69.10). In some embodiments, the rMPT comprises a functional fragment of SEQ ID NO: 104 (MPT69.10) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 104 (MPT69.10). In some embodiments, the functional fragment of SEQ ID NO: 104 (MPT69.10) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 104 (MPT69.10) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 40 (MPT4.29). In some embodiments, the rMPT comprises an amino acid sequence with at least 90% identity to SEQ ID NO: 40 (MPT4.29). In some embodiments, the rMPT comprises a functional fragment of SEQ ID NO: 40 (MPT4.29) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 40 (MPT4.29). In some embodiments, the functional fragment of SEQ ID NO: 40 (MPT4.29) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 40 (MPT4.29) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 41 (MPT4.30). In some embodiments, the rMPT comprises an amino acid sequence with at least 90% identity to SEQ ID NO: 41 (MPT4.30). In some embodiments, the rMPT comprises a functional fragment of SEQ ID NO: 41 (MPT4.30) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 41 (MPT4.30). In some embodiments, the functional fragment of SEQ ID NO: 41 (MPT4.30) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 41 (MPT4.30) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 42 (MPT4.32). In some embodiments, the rMPT comprises an amino acid sequence with at least 90% identity to SEQ ID NO: 42 (MPT4.32). In some embodiments, the rMPT comprises a functional fragment of SEQ ID NO: 42 (MPT4.32) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 42 (MPT4.32). In some embodiments, the functional fragment of SEQ ID NO: 42 (MPT4.32) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 42 (MPT4.32) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 39 (MPT4.33). In some embodiments, the rMPT comprises an amino acid sequence with at least 90% identity to SEQ ID NO: 39 (MPT4.33). In some embodiments, the rMPT comprises a functional fragment of SEQ ID NO: 39 (MPT4.33) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 39 (MPT4.33). In some embodiments, the functional fragment of SEQ ID NO: 39 (MPT4.33) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 39 (MPT4.33) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 43 (MPT4.34). In some embodiments, the rMPT comprises an amino acid sequence with at least 90% identity to SEQ ID NO: 43 (MPT4.34). In some embodiments, the rMPT comprises a functional fragment of SEQ ID NO: 43 (MPT4.34) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 43 (MPT4.34). In some embodiments, the functional fragment of SEQ ID NO: 43 (MPT4.34) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 43 (MPT4.34) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 44 (MPT4.40). In some embodiments, the rMPT comprises an amino acid sequence with at least 90% identity to SEQ ID NO: 44 (MPT4.40). In some embodiments, the rMPT comprises a functional fragment of SEQ ID NO: 44 (MPT4.40) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 44 (MPT4.40). In some embodiments, the functional fragment of SEQ ID NO: 44 (MPT4.40) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 44 (MPT4.40) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 45 (MPT4.41). In some embodiments, the rMPT comprises an amino acid sequence with at least 90% identity to SEQ ID NO: 45 (MPT4.41). In some embodiments, the rMPT comprises a functional fragment of SEQ ID NO: 45 (MPT4.41) or an amino acid sequence with at least 70%, 75%, 80%, 85%, 90%, 95%, 96%, 97%, 97.5%, 98%, 98.5%, 99%, 99.5%, 99.9%, or 100% identity to SEQ ID NO: 45 (MPT4.41). In some embodiments, the functional fragment of SEQ ID NO: 45 (MPT4.41) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 45 (MPT4.41) has at least the first 1, 2, 3, 4, 5, 6, 7, 8, 9, 10, 11, 12, 13, 14, 15, 16, 17, 18, 19, or 20 amino acids deleted from the carboxy terminus.

“Identity” refers to the extent to which the sequence of two or more nucleic acids or polypeptides is the same. In some embodiments, percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25:3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL ncbi.nlm.nih.gov for these programs. In a specific embodiment, percent identity is calculated using BLAST2 with default parameters as provided by the NCBI.

In some embodiments, the rMPT comprises a fusion domain. In some embodiments, the fusion domain improves expression and/or the overall activity of the enzyme.

In some embodiments, the rMPT is capable of converting olivetolic acid (OA) and geranyl diphosphate (GPP) to one or more products comprising cannabigerolic acid (CBGA). In some embodiments, the rMPT is capable of producing CBGA in a cell free system, in a yeast cell, in a bacterial cell, in an algae cell, or in a plant cell. In some embodiments, the one or more products comprise at least 10%, 15%, 20%, 25%, 30%, 35%, 40%, 45%, 50%, 55%, 60%, 65%, 70%, 75%, 80%, 85%, 90%, 95%, or substantially 100% CBGA. In some embodiments, at least about 50% of the one or more products is CBGA. In some embodiments, more than about 90% of the one or more products is CBGA. In some embodiments, the rMPT has a rate of formation of cannabigerolic acid (CBGA) from olivetolic acid (OA) and geranyl diphosphate (GPP) that is greater than the rate of formation of CBGA from OA and GPP by MPT4.1, MPT48 and/or MPT69 under the same conditions. In some embodiments, the rate of formation of CBGA from OA and GPP is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the rate of formation of CBGA from OA and GPP by MPT4 under the same conditions.

In some embodiments, the rMPT is capable of converting olivetolic acid (OA) and farnesyl pyrophosphate (FPP) to one or more cannabinoids, cannabinoid derivatives or cannabinoid analogues. In some embodiments, the rMPT is capable of producing cannabinoids, cannabinoid derivatives or cannabinoid analogues in a cell free system, in a yeast cell, in a bacterial cell, in an algae cell, or in a plant cell. In some embodiments, the activity of the rMPT for converting OA and FPP to one or more cannabinoids, cannabinoid derivatives or cannabinoid analogues is at least about 10%, 15%, 20%, 25%, 30%, 35%, 40%, 45%, 50%, 55%, 60%, 65%, 70%, 75%, 80%, 85%, 90%, 95%, or substantially 100% of the activity of the rMPT for converting OA and GPP to one or more cannabinoids, cannabinoid derivatives or cannabinoid analogues.

In some embodiments, the rMPT produces CBGA and FCBGA from olivetolic acid (OA) and geranyl diphosphate (GPP) and farnesyl diphosphate (FPP) respectively, at a CBGA/FCBGA ratio that is greater than the ratio of CBGA/FCBGA formation ratio from OA and GPP and FPP by MPT4.1, MPT48 or MPT69 under the same conditions. As used herein and in some embodiments, “greater than” is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the relevant control.

Recombinant rMPT with reduced or no FCBGA formation activity can be advantageous in some situations. In some embodiments, the rMPT has a rate of formation of cannabigerovarinic acid (CBGVA) from divarinic acid (DVA) and geranyl diphosphate (GPP) that is greater than the rate of formation of CBGVA from DVA and GPP by MPT4.1, MPT48, or MPT69 under the same conditions. In some embodiments, the rMPT has a ratio of CBGVA to F-CBGVA formation from DVA and GPP and FPP that is greater than the ratio of CBGVA to F-CBGVA from DVA and GPP and FPP by MPT4 under the same conditions. As used herein and in some embodiments, “greater than” is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the relevant control. In some embodiments, the rMPT does not form F-CBGVA.

In some embodiments, the rMPT has a rate of formation of CBGA from OA and GPP that is at least 1.2-fold greater (e.g., 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more) than the rate of formation of CBGA from OA and GPP by MPT4.1, MPT48, or MPT69 under the same conditions.

Cannabinoids, cannabinoid derivatives and cannabinoid analogues as recited herein are not limited. In some embodiments, cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ9-trans-tetrahydrocannabinol (Δ9-THC) type (e.g. Δ9-tetrahydrocannabinolic acid), Δ8-trans-tetrahydrocannabinol (Δ8-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), Δ9-tetrahydrocannabinolic acid A (THCA-A), Δ9-tetrahydrocannabinolic acid B (THCA-B), Δ9-tetrahydrocannabinol (THC), Δ9-tetrahydrocannabinolic acid-C4 (THCA-C4), Δ9-tetrahydrocannabinol-C4 (THC-C4), Δ9-tetrahydrocannabivarinic acid (THCVA), Δ9-tetrahydrocannabivarin (THCV), Δ9-tetrahydrocannabiorcolic acid (THCA-C1), Δ9-tetrahydrocannabiorcol (THC-C1), Δ7-cis-iso-tetrahydrocannabivarin, Δ8-tetrahydrocannabinolic acid (Δ8-THCA), Δ8-tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

In some embodiments, the rMPT is capable of converting divarinic acid (DVA) and GPP to one or more cannabinoids, cannabinoid derivatives or cannabinoid analogues. The cannabinoids are not limited and may be any disclosed herein. In some embodiments, the rMPT is capable of producing cannabinoids, cannabinoid derivatives or cannabinoid analogues in a cell free system, in a yeast cell, in a bacterial cell, in an algae cell, or in a plant cell. In some embodiments, the activity of the rMPT for converting DVA and FPP to one or more cannabinoids, cannabinoid derivatives or cannabinoid analogues is at least about 10%, 15%, 20%, 25%, 30%, 35%, 40%, 45%, 50%, 55%, 60%, 65%, 70%, 75%, 80%, 85%, 90%, 95%, or substantially 100% of the activity of the rMPT for converting OA and GPP to one or more cannabinoids, cannabinoid derivatives or cannabinoid analogues.

### Fusion Proteins

Some aspects of the present disclosure are directed to a fusion protein comprising a polypeptide having geranyl diphosphate (GPP) synthase activity and a polypeptide having prenyltransferase activity. As used herein, “GPP synthase activity” is the ability to catalyze the condensation of dimethylallyl diphosphate and isopentenyl diphosphate to geranyl diphosphate. As used herein, “prenyltransferase activity” is the ability to catalyze the transfer of a prenyl group from one compound (donor) to another (acceptor).

Some aspects of the present disclosure are directed to a fusion protein comprising a polypeptide having Geranyl diphosphate synthase activity and a polypeptide having prenyltransferase activity, wherein the polypeptide having prenyltransferase activity comprises a) a polypeptide sequence having at least 85% identity to the polypeptide sequence of MPT48 (SEQ ID NO: 47), or MPT69 (SEQ ID NO: 48), b) a polypeptide sequence comprising at least one amino acid modification compared to the amino acid sequence of MPT4.1 (SEQ ID NO: 46), or c) a polypeptide comprising i) and amino acid sequence with an at least 8 amino acid c-terminus truncation compared to a naturally occurring aromatic prenyltransferase (APT), and ii) an amino acid sequence comprising at least one amino acid modification compared to the naturally occurring APT at a position corresponding to one of the first 135 amino acids of the naturally occurring APT, or a functional fragment thereof.

In some embodiments, the polypeptide having prenyltransferase activity comprises a polypeptide sequence having at least 75%, 80%, 85%, 90%, 95%, 96%, 97%, 97.5%, 98%, 98.5%, 99%, 99.5%, or 99.9% identity to APT73.179 (SEQ ID NO: 1), APT73.119 (SEQ ID NO: 2), APT73.159 (SEQ ID NO: 3), APT73.160 (SEQ ID NO: 4), APT73.161 (SEQ ID NO: 5), APT73.162 (SEQ ID NO: 6), APT73.163 (SEQ ID NO: 7), APT73.165 (SEQ ID NO: 8), APT73.166 (SEQ ID NO: 9), APT73.168 (SEQ ID NO: 10), APT73.169 (SEQ ID NO: 11), APT73.170 (SEQ ID NO: 12), APT73.172 (SEQ ID NO: 13), APT73.182 (SEQ ID NO: 14), NO: 17), APT73.186 (SEQ ID NO: 18), APT73.187 (SEQ ID NO: 19), APT73.188 (SEQ ID NO: 20), APT73.189 (SEQ ID NO: 21), APT73.190 (SEQ ID NO: 22), APT73.191 (SEQ ID NO: 23), APT73.192 (SEQ ID NO: 24), APT73.193 (SEQ ID NO: 25), APT73.194 (SEQ ID NO: 26), APT73.195 (SEQ ID NO: 27), APT73.196 (SEQ ID NO: 28), APT73.197 (SEQ ID NO: 29), APT73.198 (SEQ ID NO: 30), APT73.199 (SEQ ID NO: 31), APT73.200 (SEQ ID NO: 32), APT73.201 (SEQ ID NO: 33), APT73.202 (SEQ ID NO: 34), APT73.203 (SEQ ID NO: 35), APT73.204 (SEQ ID NO: 36), or APT73.248 (SEQ ID NO: 96), or fragments or variants thereof.

In some embodiments, the polypeptide having prenyltransferase activity comprises a polypeptide sequence having at least 90%, 95%, 96%, 97%, 97.5%, 98%, 98.5%, 99%, or 99.9% identity to APT73.179 (SEQ ID NO: 1), APT73.119 (SEQ ID NO: 2), APT73.159 (SEQ ID NO: 3), APT73.160 (SEQ ID NO: 4), APT73.161 (SEQ ID NO: 5), APT73.162 (SEQ ID NO: 6), APT73.163 (SEQ ID NO: 7), APT73.165 (SEQ ID NO: 8), APT73.166 (SEQ ID NO: 9), APT73.168 (SEQ ID NO: 10), APT73.169 (SEQ ID NO: 11), APT73.170 (SEQ ID NO: 12), APT73.172 (SEQ ID NO: 13), APT73.182 (SEQ ID NO: 14), APT73.183 (SEQ ID NO: 15), APT73.184 (SEQ ID NO: 16), APT73.185 (SEQ ID NO: 17), APT73.186 (SEQ ID NO: 18), APT73.187 (SEQ ID NO: 19), APT73.188 (SEQ ID NO: 20), APT73.189 (SEQ ID NO: 21), APT73.190 (SEQ ID NO: 22), APT73.191 (SEQ ID NO: 23), APT73.192 (SEQ ID NO: 24), APT73.193 (SEQ ID NO: 25), APT73.194 (SEQ ID NO: 26), APT73.195 (SEQ ID NO: 27), APT73.196 (SEQ ID NO: 28), APT73.197 (SEQ ID NO: 29), APT73.198 (SEQ ID NO: 30), APT73.199 (SEQ ID NO: 31), APT73.200 (SEQ ID NO: 32), APT73.201 (SEQ ID NO: 33), APT73.202 (SEQ ID NO: 34), APT73.203 (SEQ ID NO: 35), APT73.204 (SEQ ID NO: 36), or APT73.248 (SEQ ID NO: 96), or fragments or variants thereof.

In some embodiments, the polypeptide having prenyltransferase activity comprises a polypeptide sequence having at least 75%, 80%, 85%, 90%, 95%, 96%, 97%, 97.5%, 98%, 98.5%, 99%, 99.5%, or 99.9% identity to MPT4.1 (SEQ ID NO: 46), MPT4.29 (SEQ ID NO: 40), MPT4.30 (SEQ ID NO: 41), MPT4.32 (SEQ ID NO: 42), MPT4.33 (SEQ ID NO: 40), MPT4.34 (SEQ ID NO: 43), MPT4.40 (SEQ ID NO: 44), or MPT4.41 (SEQ ID NO: 45).

In some embodiments, the polypeptide having prenyltransferase activity comprises a polypeptide sequence having at least 90%, 95%, 96%, 97%, 97.5%, 98%, 98.5%, 99%, 99.5%, or 99.9% identity to MPT4.1 (SEQ ID NO: 46), MPT4.29 (SEQ ID NO: 40), MPT4.30 (SEQ ID NO: 41), MPT4.32 (SEQ ID NO: 42), MPT4.33 (SEQ ID NO: 39), MPT4.34 (SEQ ID NO: 43), MPT4.40 (SEQ ID NO: 44), or MPT4.41 (SEQ ID NO: 45).

In some embodiments, the polypeptide having prenyltransferase activity has improved selectivity for GPP over FPP, as compared to a control membrane bound prenyltransferase or soluble aromatic prenyltransferase. In some embodiments, the polypeptide having prenyltransferase activity has an amino acid sequence comprising portions of the amino acid sequence of SEQ ID NO: 46 (MPT4.1) and SEQ ID NO: 47 (MPT48) or SEQ ID NO: 48 (MPT69). In some embodiments, the polypeptide having geranyl diphosphate synthase activity comprises a polypeptide of SEQ ID NO: 49 (GPS1.1), or a functional fragment or functional variant thereof.

In some embodiments, the fusion protein comprises a polypeptide sequence having at least 75%, 80%, 85%, 90%, 95%, 96%, 97%, 97.5%, 98%, 98.5%, 99%, 99.5%, or 99.9% identity to the polypeptide sequence of GPS1.1-L18-APT73.119 (SEQ ID NO: 52), GPS1.1-L18-APT73.159 (SEQ ID NO: 53), GPS1.1-L18-APT73.160 (SEQ ID NO: 54), GPS1.1-L18-APT73.161 (SEQ ID NO: 55), GPS1.1-L18-APT73.162 (SEQ ID NO: 56), GPS1.1-L18-APT73.163 (SEQ ID NO: 57), GPS1.1-L18-APT73.165 (SEQ ID NO: 58), GPS1.1-L18-APT73.166 (SEQ ID NO: 59), GPS1.1-L18-APT73.168 (SEQ ID NO: 60), GPS1.1-L18-APT73.169 (SEQ ID NO: 61), GPS1.1-L18-APT73.170 (SEQ ID NO: 62), GPS1.1-L18-APT73.172 (SEQ ID NO: 63), GPS1.1-L18-APT73.179 (SEQ ID NO: 64), GPS1.1-L18-APT73.182 (SEQ ID NO: 65), GPS1.1-L18-APT73.183 (SEQ ID NO: 66), GPS1.1-L18-APT73.184 (SEQ ID NO: 67), GPS1.1-L18-APT73.185 (SEQ ID NO: 68), GPS1.1-L18-APT73.186 (SEQ ID NO: 69), GPS1.1-L18-APT73.187 (SEQ ID NO: 70), GPS1.1-L18-APT73.188 (SEQ ID NO: 71), GPS1.1-L18-APT73.189 (SEQ ID NO: 72), GPS1.1-L18-APT73.190 (SEQ ID NO: 73), GPS1.1-L18-APT73.191 (SEQ ID NO: 74), GPS1.1-L18-APT73.192 (SEQ ID NO: 75), GPS1.1-L18-APT73.193 (SEQ ID NO: 76), GPS1.1-L18-APT73.194 (SEQ ID NO: 77), GPS1.1-L18-APT73.195 (SEQ ID NO: 78), GPS1.1-L18-APT73.196 (SEQ ID NO: 79), GPS1.1-L18-APT73.197 (SEQ ID NO: 80), GPS1.1-L18-APT73.198 (SEQ ID NO: 81), GPS1.1-L18-APT73.199 (SEQ ID NO: 82), GPS1.1-L18-APT73.200 (SEQ ID NO: 83), GPS1.1-L18-APT73.201 (SEQ ID NO: 84), GPS1.1-L18-APT73.202 (SEQ ID NO: 85), GPS1.1-L18-APT73.203 (SEQ ID NO: 86), GPS1.1-L18-APT73.204 (SEQ ID NO: 87), GPS1.1-L18-APT73.248 (SEQ ID NO: 97), or a functional fragment or variant thereof.

In some embodiments, the fusion protein comprises a polypeptide sequence having at least 75%, 80%, 85%, 90%, 95%, 96%, 97%, 97.5%, 98%, 98.5%, 99%, 99.5%, or 99.9% identity to GPS1.1-L11-MPT4.1 (SEQ ID NO: 88), GPS1.1-L11-MPT4.29 (SEQ ID NO: 89), GPS1.1-L11-MPT4.30 (SEQ ID NO: 90), GPS1.1-L11-MPT4.32 (SEQ ID NO: 91), GPS1.1-L11-MPT4.33 (SEQ ID NO: 92), GPS1.1-L11-MPT4.34 (SEQ ID NO: 93), GPS1.1-L11-MPT4.40 (SEQ ID NO: 94), GPS1.1-L11-MPT4.41 (SEQ ID NO: 95), or a functional fragment or variant thereof.

In some embodiments, the polypeptide having prenyltransferase activity has improved selectivity for GPP over FPP, as compared to the same unfused prenyltransferase. In some embodiments, the polypeptide having prenyltransferase activity has at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or higher selectivity for GPP over FPP as compared to the same unfused prenyltransferase. In some embodiments the polypeptide produces a CBGA and FCBGA in higher CBGA/FCBGA ratio compared to the same unfused prenyltransferase.

In some embodiments, the fusion protein further comprises a linker polypeptide between the polypeptide having Geranyl diphosphate synthase activity (GPS) and the polypeptide having prenyltransferase activity. The linker is not limited and may be any suitable linker. For example, a linker can be a short polypeptide (e.g., 15-52 amino acids). Often a linker is composed of small amino acid residues such as serine, glycine, and/or alanine. A heterologous domain could comprise a transmembrane domain, a secretion signal domain, etc. In some embodiments, the linker is a polypeptide. In some embodiments, the polypeptide is 5 to 52 amino acids in length. In some embodiments, the linker comprises a polypeptide selected from SEQ ID NO: 50 or SEQ ID NO: 51.

In some embodiments the geranyl diphosphate is fused to the N-terminus of the prenyl transferase. In other embodiments the geranyl diphosphate is fused to the C-terminus of the prenyl transferase.

### Recombinant Cells and Cell Culture

Some aspects of the present disclosure are directed to a cell expressing an rMPT as described herein. Some aspects of the present disclosure are directed to a cell having an exogenous nucleic acid sequence coding for an rMPT as described herein.

Some aspects of the present disclosure are directed to a cell comprising an engineered APT and/or rMPT, wherein the cell is capable of producing CBGA in the presence of GPP and OA. Some aspects of the present disclosure are directed to a cell comprising an engineered APT and/or rMPT, wherein the cell is capable of producing CBGVA in the presence of GPP and DVA. Some aspects of the present disclosure are directed to a cell comprising an APT and/or rMPT, wherein the cell is capable of making a cannabinoid in the presence of a carbon source and, optionally, hexanoic or butyric acid.

In some embodiments, the cell is a yeast cell or a bacterial cell. In some embodiments, the yeast cell is a Yarrowia strain or a Saccharomyces strain.

Some aspects of the present disclosure are directed to a method of producing CBGA, CBGVA or derivatives thereof comprising culturing a cell comprising an APT or rMPT under suitable conditions to produce CBGA, CBGVA or derivatives thereof. In some embodiments, the suitable condition comprises supplementing a culture media in which the cell is cultured with at least one of butyric acid, valeric acid, isovaleric acid, hexanoic acid, hexanol, butanol, oleic acid, glycerol or glucose.

Some aspects of the present disclosure are directed to a cell expressing a fusion protein as described herein. Some aspects of the present disclosure are directed to a cell having an exogenous nucleic acid sequence coding for a fusion protein as described herein.

Some aspects of the present disclosure are directed to a cell comprising a fusion protein, wherein the cell is capable of producing CBGA in the presence of OA and GPP. Some aspects of the present disclosure are directed to a cell comprising a fusion protein, wherein the cell is capable of producing CBGVA in the presence of DVA and a GPP. Some aspects of the present disclosure are directed to a cell comprising a fusion protein, wherein the cell is capable of making a cannabinoid in the presence of a carbon source and, optionally, hexanoic or butyric acid.

In some embodiments, the cell is capable of forming acyl-CoA from a carboxylic acid. In some embodiments, the cell encodes an exogenous hexanoyl-CoA synthetase and/or butyryl-CoA synthetase.

In some embodiments, the cell is a yeast cell or a bacterial cell. In some embodiments, the yeast cell is a Yarrowia strain or a Saccharomyces strain.

Some aspects of the present disclosure are directed to a method of producing CBGA, CBGVA or derivatives thereof comprising culturing a cell comprising a fusion protein under suitable conditions to produce CBGA, CBGVA or derivatives thereof. In some embodiments, the suitable condition comprises supplementing a culture media in which the cell is cultured with at least one of butyric acid, valeric acid, isovaleric acid, hexanoic acid, hexanol, butanol, oleic acid, glycerol or glucose.

The cell is not limited and may be any suitable cell for expression. In some embodiments, the cell may be a microorganism or a plant. In some embodiments, the microorganism is a bacteria (e.g., E. Coli), an algae, or a yeast. In some embodiments, the yeast is an oleaginous yeast (e.g., a Yarrowia lipolytica strain). In some embodiments, the bacteria is Escherichia coli.

Suitable cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha (now known as Pichia angusta), Kluyveromyces sp., Kluyveromyces lactis, Kluyveromyces marxianus, Schizosaccharomyces pompe, Dekkera bruxellensis, Arxula adeninivorans, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, Yarrowia lipolytica and the like. In some embodiments, the cell is a protease-deficient strain of Saccharomyces cerevisiae. In some embodiments, the cell is a eukaryotic cell other than a plant cell. In some embodiments, the cell is a plant cell. In some embodiments, the cell is a plant cell, where the plant cell is one that does not normally produce a cannabinoid, a cannabinoid derivative or analogue, a cannabinoid precursor, or a cannabinoid precursor derivative or analogue. In some embodiments, the cell is Saccharomyces cerevisiae. In some embodiments, the cell disclosed herein is cultured in vitro.

In some embodiments, the cell is a prokaryotic cell. Suitable prokaryotic cells may include, but are not limited to, any of a variety of laboratory strains of Escherichia coli, Lactobacillus sp., Salmonella sp., Shigella sp., and the like. See, e.g., Carrier et al, (1992) J. Immunol. 148:1176-1181; U.S. Pat. No. 6,447,784; and Sizemore et al. (1995) Science 270:299-302. Examples of Salmonella strains which can be employed may include, but are not limited to, Salmonella typhi and S. typhimurium. Suitable Shigella strains may include, but are not limited to, Shigella flexneri, Shigella sonnei, and Shigella disenteriae. Typically, the laboratory strain is one that is non-pathogenic. Non-limiting examples of other suitable bacteria may include, but are not limited to, Bacillus subtilis, Pseudomonas putida, Pseudomonas aeruginosa, Pseudomonas mevalonii, Rhodobacter sphaeroides, Rhodobacter capsulatus, Rhodospirillum rubrum, Rhodococcus sp., and the like.

An expression vector or vectors can be constructed to include exogenous nucleotide sequences coding for the rMPT or APT described herein operably linked to expression control sequences functional in the cell. Expression vectors applicable include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “exogenous” is intended to mean that the referenced molecule or the referenced activity is introduced into the cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. When used in reference to a biosynthetic activity, the term refers to an activity that is introduced into the host. The source can be, for example, a homologous or heterologous encoding nucleic acid that expresses the referenced activity following introduction into the cell. Therefore, the term “endogenous” refers to a referenced molecule or activity that is present in the cell. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

In some embodiments, the cell expressing rMPT or APT is capable of producing CBGA in the presence of GPP and OA (e.g., as metabolically produced by the cell or through the presence of OA). In some embodiments, the cell expressing rMPT or APT is capable of producing CBGA from a carbon source. In some embodiments, the cell expressing rMPT or APT is capable of producing CBGA from a carbon source in the presence of hexanoic acid.

In some embodiments, the cell expressing rMPT or APT is capable of producing CBGVA in the presence of GPP and DVA (e.g., as metabolically produced by the cell or through the presence of one or more of DVA in the media). In some embodiments, the cell expressing rMPT or APT is capable of producing CBGVA from a carbon source. In some embodiments, the cell expressing rMPT or APT is capable of producing CBGVA from a carbon source in the presence of butyric acid.

In some embodiments, the cell expressing rMPT or APT is capable of making a cannabinoid or analog thereof in the presence of a carbon source and, optionally, hexanoic or butyric acid.

In some embodiments, the cell expressing the fusion protein is capable of producing CBGA in the presence of GPP and OA (e.g., as metabolically produced by the cell or through the presence of OA in the media). In some embodiments, the cell expressing the fusion protein is capable of producing CBGA from a carbon source. In some embodiments, the cell expressing the fusion protein is capable of producing CBGA from a carbon source in the presence of hexanoic acid.

In some embodiments, the cell expressing the fusion protein is capable of producing CBGVA in the presence of GPP and DVA (e.g., as metabolically produced by the cell or through the presence of DVA in the media). In some embodiments, the cell expressing the fusion protein is capable of producing CBGVA from a carbon source. In some embodiments, the cell expressing the fusion protein is capable of producing CBGVA from a carbon source in the presence of butyric acid.

In some embodiments, the cell expressing the fusion protein is capable of making a cannabinoid or analog thereof in the presence of a carbon source and, optionally, hexanoic or butyric acid. Exemplary carbon sources include sugar carbons such as sucrose, glucose, mannitol, galactose, fructose, mannose, isomaltose, xylose, pannose, maltose, arabinose, cellobiose and 3-, 4-, or 5-oligomers thereof. Other carbon sources include alcohol carbon sources such as, ethanol, glycerol. Other carbon sources may contain a combination of the above carbon sources such as, for example, glucose/mannitol or glucose/ethanol. Other carbon sources include acid and esters such as acetate or formate, or fatty acids having four to twenty-two carbon atoms or fatty acid esters thereof. Other carbon sources can include renewal feedstocks and biomass. Exemplary renewal feedstocks include cellulosic biomass, hemicellulosic biomass and lignin feedstocks. Mixed carbon sources can also be used, such as a fatty acid and a sugar as described herein.

Depending on the cell, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

The culture conditions can include, for example, liquid culture procedures as well as fermentation and other large-scale culture procedures. Useful yields of the products can be obtained under aerobic culture conditions. An exemplary growth condition for achieving, one or more cannabinoid products includes aerobic culture or fermentation conditions. In certain embodiments, the microbial organism can be sustained, cultured or fermented under aerobic conditions.

Substantially aerobic conditions include, for example, a culture, batch fermentation or continuous fermentation such that the dissolved oxygen concentration in the medium remains between 5% and 100% of saturation. The percent of dissolved oxygen can be maintained by, for example, sparging air, pure oxygen or a mixture of air and oxygen.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

In some embodiments, the method comprises providing a cell as described herein comprising an exogenous nucleotide sequence coding for a rMPT or APT as described herein and culturing the cell to produce a cannabinoid, cannabinoid derivative, or cannabinoid analogue thereof.

In some embodiments, the method comprises providing a cell as described herein comprising an exogenous nucleotide sequence coding for a fusion protein described herein and culturing the cell to produce a cannabinoid, cannabinoid derivative, or cannabinoid analogue thereof. In some embodiments, the method comprises providing a cell as described herein comprising an exogenous nucleotide sequence coding for a fusion protein described herein and culturing the cell to produce the cannabinoid or analogue thereof.

In some embodiments, the method comprises providing a cell as described herein comprising an exogenous nucleotide sequence coding for an OA or DVA importer protein described herein and culturing the cell to produce a cannabinoid, cannabinoid derivative, or cannabinoid analogue thereof. In some embodiments, the method comprises providing a cell as described herein comprising an exogenous nucleotide sequence coding for an OA or DVA importer protein described herein and culturing the cell to produce the cannabinoid or analogue thereof.

In some embodiments, the method comprises providing a cell as described herein comprising an inactivated ore deleted nucleotide sequence coding for an OA or DVA exporter protein described herein and culturing the cell to produce a cannabinoid, cannabinoid derivative, or cannabinoid analogue thereof. In some embodiments, the method comprises providing a cell as described herein comprising an inactivated or deleted nucleotide sequence coding for an OA or DVA exporter protein described herein and culturing the cell to produce the cannabinoid or analogue thereof.

The cannabinoids, cannabinoid derivatives and cannabinoid analogues produced by the methods disclosed herein are not limited and may be any disclosed cannabinoid. In some embodiments, the cannabinoids, cannabinoid derivatives and cannabinoid analogues are selected from cannabigerolic acid, tetrahydrocannabinolic acid, tetrahydrocannabinol, cannabidiolic acid, cannabidiol, cannabigerol, cannabichromenic acid, cannabichromene, or an acid or derivative or analogue thereof.

In some embodiments, the methods further comprise a step of purifying or isolating the cannabinoids, derivatives or analogues thereof from the culture. Methods of isolation are not limited and may be any suitable method known in the art. Purification methods include, for example, extraction procedures (e.g., using supercritical carbon dioxide, ethanol or mixtures of these two), as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration or centrifugal partition chromatography (CPC).

In some embodiments, the cells are grown in stirred tank fermenters with feed supplementation (sugars with or without organic acids) where the dissolved oxygen, temperature, and pH are be controlled according to the optimal growth and production process. In some embodiments, aqueous non-miscible organic solvents are supplemented to dissolve added organic acids or extract the cannabinoid products as they are being synthesized. In some embodiments, these solvents may include, but are not limited to, isopropyl myristate (IPM), diisobutyl adipate, Bis (2-ethylhexyl) adipate, decane, dodecane, hexadecane or anther organic solvent with logP>5. The later number (logP) is defined as the log of a compound's partition between water and octanol and is a standard parameter of a compound's hydrophobicity (the larger the logP the less soluble in water). Depending on the fermentation process, the products can be isolated and purified using different methods.

If no organic cosolvent is used the targeted cannabinoid(s) precipitate together with the cell biomass after centrifugation, or is isolated in the solids after water removal using spray drying or other methods that remove water (i.e lyophilization, ultrafiltration etc). In one embodiment, an aqueous miscible organic solvent (ethanol, acetonitrile, etc.) is added to the cannabinoid containing cell pellet to dissolve the products. In some embodiments, a simple filtration, ultrafiltration or centrifugation can remove the cells and the aqueous/organic media evaporated to dryness or to a small volume from which the cannabinoid product will precipitate or crystalize. Alternatively, the cannabinoid containing cell pellet can be extracted with an aqueous immiscible organic solvent (ethyl acetate, heptane, decane, etc.) or supercritical carbon dioxide (with 0-10% Ethanol) to extract the cannabinoids. Evaporation of the organic solvent and a possible recrystallization will produce pure cannabinoid. If the cannabinoid products are not extracted by the previous methods and are trapped inside the cell, cells lysis may be required prior to extraction methods described above. In some embodiments, cells are disrupted using mechanical methods or by suspension in appropriate lysis buffers from which the cannabinoids can be extracted with an organic aqueous immiscible solvent (ethyl acetate, hexane, decane, methylene chloride, etc.). In other embodiments, cells may be suspended in an organic solvent (ethanol, methanol, methylene chloride, etc.) that extracts the cannabinoids from the cells.

In some embodiments, an organic solvent is required during growth that is separated at the end of the fermentation. Back extraction with alkaline aqueous solvent or a different organic solvent with low boiling point and high polarity (ethanol, acetonitrile, etc.) will remove the cannabinoids. Isolation can then involve a simple pH shift if water is used, or an evaporation if organic solvents are used. In both cases, a recrystallization step may be required at the end to improve purity of the product.

### EXAMPLES

### Example 1: Activity of MPT4 and APT73 Mutants

Plasmids pYE01.GPS1.1.L18.APT73.119, pYE01.GPS1.1.L11.MPT4.1, and variants of each were transformed into strain SB-1334. Multiple colonies per transformation were precultured for 24 h in YNB containing glucose (6%), casamino acids (1%), MES (100 mM pH 6.5), and hygromycin (1 mg/mL) at 30° C. with 1000 rpm shaking. Pre-cultures were each used to inoculate two separate cultures with assay medium comprised of YNB containing glucose (6%), casamino acids (1%), MES (100 mM pH 6.5), and hygromycin (1 mg/mL). Of these two separate cultures, one was supplemented with 5 mM OA after 24 h and one was supplemented with 5 mM DVA after 24 h. Cultures (0.5 mL) were incubated in a 96 well block at 30° C. with 1000 rpm shaking and were quenched with equal volumes of EtOH after 48 h (OA) or 72 h (DVA) total growth. Samples were analyzed by HPLC/MS and the main products CBG(V)A are shown in the following tables. Averages and standard deviations were calculated from replicates. No FCBG(V) A was detected.

TABLE 1
Product concentration in μM (micromolar)

| | | CBGA | CBGVA |
|---|---|---|---|
| | Enzyme | (OA | (DVA |
| Strain | Expressed | Feed) | Feed) |
| SB-1334 + | GPS1.1-L11- | 68 ± 1 | 22 ± 1 |
| pYE01.GPS1.1.L11.MPT4.1 | MPT4.1 | | |
| SB-1334 + | GPS1.1-L11- | 86 ± 2 | 42 ± 14 |
| pYE01.GPS1.1.L11.MPT4.29 | MPT4.29 | | |
| SB-1334 + | GPS1.1-L11- | 105 ± 5 | 52 ± 11 |
| pYE01.GPS1.1.L11.MPT4.30 | MPT4.30 | | |
| SB-1334 + | GPS1.1-L11- | 109 ± 3 | 37 ± 2 |
| pYE01.GPS1.1.L11.MPT4.32 | MPT4.32 | | |
| SB-1334 + | GPS1.1-L11- | 119 ± 3 | 34 ± 3 |
| pYE01.GPS1.1.L11.MPT4.33 | MPT4.33 | | |
| SB-1334 + | GPS1.1-L11- | 80 ± 15 | 34 ± 5 |
| pYE01.GPS1.1.L11.MPT4.34 | MPT4.34 | | |
| SB-1334 + | GPS1.1-L11- | 77 ± 3 | 33 ± 3 |
| pYE01.GPS1.1.L11.MPT4.40 | MPT4.40 | | |
| SB-1334 + | GPS1.1-L11- | 110 ± 4 | 42 ± 4 |
| pYE01.GPS1.1.L11.MPT4.41 | MPT4.41 | | |
| SB-1334 + | GPS1.1-L18- | 216 ± 7 | 75 ± 13 |
| pYE01.GPS1.1.L18.APT73.119 | APT73.119 | | |
| SB-1334 + | GPS1.1-L18- | 229 ± 5 | 108 ± 10 |
| pYE01.GPS1.1.L18.APT73.159 | APT73.159 | | |
| SB-1334 + | GPS1.1-L18- | 225 ± 6 | 108 ± 26 |
| pYE01.GPS1.1.L18.APT73.160 | APT73.160 | | |
| SB-1334 + | GPS1.1-L18- | 213 ± 7 | 105 ± 12 |
| pYE01.GPS1.1.L18.APT73.161 | APT73.161 | | |
| SB-1334 + | GPS1.1-L18- | 222 ± 3 | 125 ± 6 |
| pYE01.GPS1.1.L18.APT73.162 | APT73.162 | | |
| SB-1334 + | GPS1.1-L18- | 141 ± 2 | 131 ± 31 |
| pYE01.GPS1.1.L18.APT73.163 | APT73.163 | | |
| SB-1334 + | GPS1.1-L18- | 192 ± 6 | 224 ± 24 |
| pYE01.GPS1.1.L18.APT73.165 | APT73.165 | | |
| SB-1334 + | GPS1.1-L18- | 220 ± 8 | 127 ± 10 |
| pYE01.GPS1.1.L18.APT73.166 | APT73.166 | | |
| SB-1334 + | GPS1.1-L18- | 217 ± 6 | 116 ± 9 |
| pYE01.GPS1.1.L18.APT73.168 | APT73.168 | | |
| SB-1334 + | GPS1.1-L18- | 199 ± 3 | 114 ± 8 |
| pYE01.GPS1.1.L18.APT73.169 | APT73.169 | | |
| SB-1334 + | GPS1.1-L18- | 192 ± 4 | 109 ± 23 |
| pYE01.GPS1.1.L18.APT73.170 | APT73.170 | | |
| SB-1334 + | GPS1.1-L18- | 20 ± 4 | 111 ± 9 |
| pYE01.GPS1.1.L18.APT73.172 | APT73.172 | | |
| SB-1334 + | GPS1.1-L18- | 319 ± 10 | 146 ± 16 |
| pYE01.GPS1.1.L18.APT73.179 | APT73.179 | | |

### Example 2: Activity of MPT48 and MPT69

Plasmids pYE01.MPT48 and pYE01.MPT69, and variants of each were transformed into strains SB-1459-5.2 and SB-1714-5.3. Multiple colonies per transformation were precultured for 24 h in YNB containing glucose (6%), casamino acids (1%), MES (100 mM pH 6.5), and hygromycin (1 mg/mL) at 30° C. with 1000 rpm shaking. Pre-cultures were each used to inoculate two separate cultures with assay medium comprised of YNB containing glucose (6%), casamino acids (1%), MES (100 mM pH 6.5), and hygromycin (1 mg/mL). Of these two separate cultures, one contained 2 mM OA and one contained 5 mM DVA. Cultures (0.5 mL) were incubated in a 96 well block at 30° C. with 1000 rpm shaking and were quenched with equal volumes of EtOH after 38 h (OA) or 48 h (DVA) total growth. Samples were analyzed by HPLC/MS and the main products CBG (V) A and FCBG (V) A are shown in the following tables. Averages and standard deviations were calculated from replicates.

TABLE 2
Product concentration in μM (micromolar)

| | Enzyme | CBGA | FCBGA | CBGVA | FCBGVA |
|---|---|---|---|---|---|
| | Ex- | (OA | (OA | (DVA | (DVA |
| Strain | pressed | Feed) | Feed) | Feed) | Feed) |
| SB-1459-5.2 + | MPT48 | 979 ± 18 | 159 ± 2 | 91 ± 6 | 481 ± 10 |
| pYE01.MPT48 | | | | | |
| SB-1459-5.2 + | MPT69 | 772 ± 90 | 197 ± 44 | 13 ± 4 | 189 ± 69 |
| pYE01.MPT69 | | | | | |
| SB-1714-5.3 + | MPT48 | 298 ± 7 | 886 ± 15 | 42 ± 1 | 2570 ± 31 |
| pYE01.MPT48 | | | | | |
| SB-1714-5.3 + | MPT69 | 371 ± 23 | 956 ± 32 | 34 ± 2 | 2248 ± 42 |
| pYE01.MPT69 | | | | | |

TABLE 3
Genes expressed

| Strain | Key genes expressed |
|---|---|
| SB-1334 | GPS1.1, HCS2, GPS1.A28, PKC1, PKS1.1 |
| SB-1459-5.2 | GPS1.1, HCS2, GPS1.A28, PKC1, PKS1.1, ΔACD1 |
| SB-1714-5.3 | GPS1.1, HCS2, MvaE, PKS1.1, PKC1 |

The enzymes are screened in two different Yarrowia strains. One contains the native Erg20 in the genome (SB-1714-5.3) while the other strain (SB-1459-5.2) is engineered to contain the Erg20.A28 mutation (overexpression ERG20.F88W.N119W, expression of ERG20.A28 allele, and disruption of the endogenous Erg20 as described in co-owned PCT Application No. PCT/US2022/046926. As expected the Δ28 strain that produces increased amounts of GPP over FPP, improves the CBG (V) A/FCBG (V) A ratio particularly when OA is the prenylation substrate.

Mutagenesis of these enzymes as described herein, will improve the CBG (V) A to FCBG (V) A ratio and also increase the overall activity.

### Example 3: Activity of APT73 Mutants

Plasmids pYE01.GPS1.1.L18.APT73.165 and variants were transformed into strain SB-1334. Multiple colonies per transformation were precultured for 24 h in YNB containing glucose (6%), casamino acids (1%), MES (100 mM pH 6.5), and hygromycin (1 mg/mL) at 30° C. with 1000 rpm shaking. Pre-cultures were each used to inoculate two separate cultures with assay medium comprised of YNB containing glucose (6%), casamino acids (1%), MES (100 mM pH 6.5), and hygromycin (1 mg/mL). Of these two separate cultures, one was supplemented with 5 mM OA after 24 h and one was supplemented with 5 mM DVA after 24 h. Cultures (0.5 mL) were incubated in a 96 well block at 30° C. with 1000 rpm shaking and were quenched with equal volumes of EtOH after 40 h total growth. Samples were analyzed by HPLC/MS and the main products CBG (V) A are shown in the following tables. Averages and standard deviations were calculated from replicates. No FCBG (V) A was detected.

TABLE 4
Product concentration in μM (micromolar)

| | | CBGA | CBGVA |
|---|---|---|---|
| | Enzyme | (OA | (DVA |
| Strain | Expressed | Feed) | Feed) |
| SB-1334 + | GPS1.1-L18- | 132 ± 9 | 79 ± 4 |
| pYE01.GPS1.1.L18.APT73.165 | APT73.165 | | |
| SB-1334 + | GPS1.1-L18- | 136 ± 28 | 86 ± 4 |
| pYE01.GPS1.1.L18.APT73.182 | APT73.182 | | |
| SB-1334 + | GPS1.1-L18- | 137 ± 22 | 100 ± 5 |
| pYE01.GPS1.1.L18.APT73.183 | APT73.183 | | |
| SB-1334 + | GPS1.1-L18- | 147 ± 29 | 88 ± 6 |
| pYE01.GPS1.1.L18.APT73.184 | APT73.184 | | |
| SB-1334 + | GPS1.1-L18- | 137 ± 26 | 106 ± 8 |
| pYE01.GPS1.1.L18.APT73.185 | APT73.185 | | |
| SB-1334 + | GPS1.1-L18- | 133 ± 20 | 101 ± 6 |
| pYE01.GPS1.1.L18.APT73.186 | APT73.186 | | |
| SB-1334 + | GPS1.1-L18- | 130 ± 19 | 102 ± 4 |
| pYE01.GPS1.1.L18.APT73.187 | APT73.187 | | |
| SB-1334 + | GPS1.1-L18- | 139 ± 29 | 104 ± 7 |
| pYE01.GPS1.1.L18.APT73.188 | APT73.188 | | |
| SB-1334 + | GPS1.1-L18- | 139 ± 24 | 80 ± 3 |
| pYE01.GPS1.1.L18.APT73.189 | APT73.189 | | |
| SB-1334 + | GPS1.1-L18- | 150 ± 23 | 90 ± 4 |
| pYE01.GPS1.1.L18.APT73.190 | APT73.190 | | |
| SB-1334 + | GPS1.1-L18- | 163 ± 10 | 93 ± 4 |
| pYE01.GPS1.1.L18.APT73.191 | APT73.191 | | |
| SB-1334 + | GPS1.1-L18- | 136 ± 5 | 98 ± 5 |
| pYE01.GPS1.1.L18.APT73.192 | APT73.192 | | |
| SB-1334 + | GPS1.1-L18- | 225 ± 22 | 92 ± 8 |
| pYE01.GPS1.1.L18.APT73.193 | APT73.193 | | |
| SB-1334 + | GPS1.1-L18- | 174 ± 8 | 79 ± 7 |
| pYE01.GPS1.1.L18.APT73.196 | APT73.196 | | |
| SB-1334 + | GPS1.1-L18- | 176 ± 11 | 95 ± 11 |
| pYE01.GPS1.1.L18.APT73.197 | APT73.197 | | |
| SB-1334 + | GPS1.1-L18- | 171 ± 11 | 87 ± 7 |
| pYE01.GPS1.1.L18.APT73.199 | APT73.199 | | |
| SB-1334 + | GPS1.1-L18- | 170 ± 15 | 88 ± 6 |
| pYE01.GPS1.1.L18.APT73.200 | APT73.200 | | |

### Example 4: Activity of APT73.187 and MPT69 Mutants

Plasmids pYE01.GPS1.1.L18.APT73.187 and pYE01.GPS1.1.L18.APT73.248 were transformed into strain SB-3589. Multiple colonies per transformation were precultured for 48 h in YNB containing glucose (6%), casamino acids (1%), MES (100 mM pH 6.5), and hygromycin (1 mg/mL) at 30° C. with 1000 rpm shaking. Pre-cultures were each used to inoculate two separate cultures with assay medium comprised of YNB containing glucose (6%), casamino acids (1%), MES (100 mM pH 6.5), and hygromycin (1 mg/mL). Of these two separate cultures, one was supplemented with 2 mM OA after 24 h and one was supplemented with 2 mM DVA after 24 h. Cultures (0.5 mL) were incubated in a 96 well block at 30° C. with 1000 rpm shaking and were quenched with equal volumes of EtOH after 48 h total growth. Samples were analyzed by HPLC/MS and the main products CBG (V) A+THC (V) A (THCAS expressed from the genome converts some CBG (V) A to THC (V) A) are shown in the following tables. Averages and standard deviations were calculated from replicates. No FCBGVA and only trace amounts (<2% total products) of FCBGA were detected.

TABLE 5
Product concentration in μM (micromolar)

| | Enzyme | CBGA + THCA | CBGVA + THCVA |
|---|---|---|---|
| Strain | Expressed | (OA Feed) | (DVA Feed) |
| SB-3589 + | GPS1.1-L18- | 1415 ± 183 | 1071 ± 56 |
| pYE01.GPS1.1.L18.APT73.187 | APT73.187 | | |
| SB-3589 + | GPS1.1-L18- | 2085 ± 53 | 1770 ± 107 |
| pYE01.GPS1.1.L18.APT73.248 | APT73.248 | | |

Plasmids pYE01.MPT69 and variants were transformed into strain SB-3589. Multiple colonies per transformation (except for transformations of pYE01.MPT69.6, pYE01.MPT69.9, and pYE01.MPT69.10) were precultured for 48 h in YNB containing glucose (6%), casamino acids (1%), MES (100 mM pH 6.5), and hygromycin (1 mg/mL) at 30° C. with 1000 rpm shaking. Pre-cultures were each used to inoculate cultures with assay medium comprised of YNB containing glucose (6%), casamino acids (1%), MES (100 mM pH 6.5), and hygromycin (1 mg/mL) which were supplemented with 2 mM DVA after 24 h. Cultures (0.5 mL) were incubated in a 96 well block at 30° C. with 1000 rpm shaking and were quenched with equal volumes of EtOH after 48 h total growth. Samples were analyzed by HPLC/MS and the main products CBGVA+THCVA (THCAS expressed from the genome converts some CBGVA to THCVA) and FCBGVA are shown in the following tables. Averages and standard deviations were calculated from replicates.

TABLE 6
Product concentration in μM (micromolar)

| | Enzyme | CBGVA + | |
|---|---|---|---|
| Strain | Expressed | THCVA | FCBGVA |
| SB-3589 + pYE01.MPT69 | MPT69 | 239 ± 32 | 172 ± 23 |
| SB-3589 + pYE01.MPT69.2 | MPT69.2 | 317 ± 11 | 58 ± 1 |
| SB-3589 + pYE01.MPT69.5 | MPT69.5 | 236 ± 48 | 57 ± 4 |
| SB-3589 + pYE01.MPT69.6 | MPT69.6 | 290 | 135 |
| SB-3589 + pYE01.MPT69.7 | MPT69.7 | 346 ± 77 | 166 ± 32 |
| SB-3589 + pYE01.MPT69.8 | MPT69.8 | 402 ± 72 | 156 ± 9 |
| SB-3589 + pYE01.MPT69.9 | MPT69.9 | 321 | 102 |
| SB-3589 + pYE01.MPT69.10 | MPT69.10 | 266 | 131 |

TABLE 7
Genes expressed

| Strain | Key genes expressed |
|---|---|
| SB-3589 | GPS1.1, HCS2, GPS1.A28, THCAS, CNE1, tHMGR |

### EXPERIMENTAL METHODS

### Cloning Methods, Vectors and Strains

### Yarrowia Expression Plasmids

Genes for each enzyme were optimized for expression in Yarrowia, synthesized (Codex DNA), and cloned into pYE01 vector. Plasmids were transformed into chemically competent E. coli NEB 10-beta cells (NEB), plated on LB agar plates with 50 μg/mL kanamycin, and grown overnight at 37° C. Colony PCR was used to verify gene fragment insertion and positive colonies were inoculated into liquid LB media with 50 μg/mL kanamycin. Cultures were grown overnight at 37° C. and then used for isolating plasmid DNA (Qiagen).

### Analytical Methods

All samples after quenching with equal volume of EtOH were centrifuged and were analyzed by HPLC-MS

All samples were quenched with equal volume of EtOH containing 0.2 mg/mL internal standard (3,5-Diisopropyl-2-hydroxybenzoic acid CAS #2215-21-6) centrifuged, and clarified solutions were analyzed by HPLC-MS

### Method A

Column: 2.1×50 mm COSMOCORE PBr (Nacalai USA, Inc.)
Mobile Phase: A; 0.1% formic acid in water, B; 0.1% formic acid in acetonitrile
Flow: 0.45 mL/min
Temp: % 50 Celsius
Gradient: 20% B at 0 min, 70% B at 2.3 min, 89% B at 4.2 min, 20% B at 4.3 min, 20% B at 6 min

TABLE 8
Detection: UV DAD and QToF MS

| Compound | Retention Time (min) |
|---|---|
| Butyric acid | 0.60 |
| Hexanoic acid | 1.53 |
| DOL (Divarinol) | 1.65 |
| DVA (Divarinic acid) | 1.89 |
| OL (Olivetol) | 2.32 |
| OA (Olivetolic acid) | 2.43 |
| Internal Std. | 3.09 |
| CBGVA | 3.57 |
| CBGA | 3.89 |
| F-CBGVA | 4.27 |
| F-CBGA | 4.58 |

All compounds were confirmed based on authentic standards, by retention time, UV profile and MS comparisons. For FCBGA and FCBGA no authentic standards were available so these compounds were identified based on UV profile, and MS analysis (molecular ion and fragmentation pattern).

### Process Development for Making Cannabinoids Through Fermentation

The above CBGA synthases can be used in cell free reactions (in vitro) to produce CBGA and analogs by the feeding of the appropriate substrates or can be introduced into a recombinant organism (yeast, bacteria, fungus, algae, or plant) to improve the flux towards CBGA or any of its analogs. These recombinant organisms will contain the optimized genes described herein to synthesize olivetolic acid and CBGA (or their analogs) engineered mevalonate or MEP pathway to increase flux towards GPP or FPP. To improve flux and increase the intracellular concentration of GPP, mutant farnesyl pyrophosphate synthases may be used as have been described in yeast (Jian G-Z, et al Metabolic Engineering, 2017, 41, 57) or GPP specific synthases can be introduced (Schmidt A, Gershenzon J. Phytochemistry, 2008, 69, 49). Other enzymes in the mevalonate pathway (for example HMG-CoA reductase) may need to be manipulated (truncated or mutated) or be overexpressed. The formation of GPP/FPP and OA can occur when the organism is grown with simple carbon sources, such as glucose, sucrose, glycerol, or another simple or complex sugar mixture. External organic acids with carbon chains varying from 4 to more than 12 (in straight or branched chains) can also be supplemented during growth. With supplementation, introduction of the appropriate acid-CoA synthase may be required to produce the corresponding organic acid-CoAs that can then be used by PKS and PKC to produce OA analogs. The organism can also express the appropriate synthase that cyclizes CBGA or any of its analogs to other cannabinoids. A description of these enzymes is described in more detail in co-owned PCT application PCT/US22/46926 hereby incorporated herein by reference in its entirety.

LISTING OF AMINIO ACID SEQUENCES
MUTANT AND NATURAL (APT73) SOLUBLE AROMATIC
PRENYLTRANSFERASE ENZYMES
APT73.179
(SEQ ID NO: 1)
MDEVYAAVEQTSRLLDVPCSPDRFEPVWKAFGDQLPDSVLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKGRRCLDG
APT73.119
(SEQ ID NO: 2)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKGRRCLDG
APT73.159
(SEQ ID NO: 3)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTNQPGELPAPHDEPT
EAFARQVPHVYEGGREFVSGVALAPSGASYYKLAALYQKGRRCLDG
APT73.160
(SEQ ID NO: 4)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGPREFVSGVALAPSGASYYKLAALYQKGRRCLDG
APT73.161
(SEQ ID NO: 5)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGVREFVSGVALAPSGASYYKLAALYQKGRRCLDG
APT73.162
(SEQ ID NO: 6)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGSREFVSGVALAPSGASYYKLAALYQKGRRCLDG
APT73.163
(SEQ ID NO: 7)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAAIYQKGRRCLDG
APT73.165
(SEQ ID NO: 8)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAAIYQKGRRCLDG
APT73.166
(SEQ ID NO: 9)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKGRKCLDG
APT73.168
(SEQ ID NO: 10)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKGRRELDG
APT73.169
(SEQ ID NO: 11)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKGRRPLDG
APT73.170
(SEQ ID NO: 12)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKGRRCLDGRYG
APT73.172
(SEQ ID NO: 13)
MDEVYAAVEQTSRLLDVPCSPDRFEPVWKAFGDQLPDSHLCFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKGRRCLDGHRR
APT73.182
(SEQ ID NO: 14)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGPREFVSGVALAPSGASYYKLAAIYQKGRRCLDG
APT73.183
(SEQ ID NO: 15)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEGPREFVSGVALAPSGASYYKLAAIYQKGRRPLDG
APT73.184
(SEQ ID NO: 16)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGVREFVSGVALAPSGASYYKLAAIYQKGRRCLDG
APT73.185
(SEQ ID NO: 17)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEGVREFVSGVALAPSGASYYKLAAIYQKGRRPLDG
APT73.186
(SEQ ID NO: 18)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGGREFVSGVALAPSGASYYKLAAIYQKGRRPLDG
APT73.187
(SEQ ID NO: 19)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGPREFVSGVALAPSGASYYKLAAIYQKGRRPLDG
APT73.188
(SEQ ID NO: 20)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGVREFVSGVALAPSGASYYKLAAIYQKGRRPLDG
APT73.189
(SEQ ID NO: 21)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGGREFVSGVALAPSGASYYKLAAIYQKGRRCLDG
APT73.190
(SEQ ID NO: 22)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEPGREFVSGVALAPSGASYYKLAAIYQKGRRCLDG
APT73.191
(SEQ ID NO: 23)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEVGREFVSGVALAPSGASYYKLAAIYQKGRRCLDG
APT73.192
(SEQ ID NO: 24)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAAIYQKGRRPLDG
APT73.193
(SEQ ID NO: 25)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAAIYQKGRRCLDG
APT73.194
(SEQ ID NO: 26)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEPGREFVSGVALAPSGASYYKLAAIYQKGRRCLDG
APT73.195
(SEQ ID NO: 27)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEPGREFVSGVALAPSGASYYKLAAIYQKGRRPLDG
APT73.196
(SEQ ID NO: 28)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEVGREFVSGVALAPSGASYYKLAAIYQKGRRCLDG
APT73.197
(SEQ ID NO: 29)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEVGREFVSGVALAPSGASYYKLAAIYQKGRRPLDG
APT73.198
(SEQ ID NO: 30)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGGREFVSGVALAPSGASYYKLAAIYQKGRRPLDG
APT73.199
(SEQ ID NO: 31)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEPGREFVSGVALAPSGASYYKLAAIYQKGRRPLDG
APT73.200
(SEQ ID NO: 32)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEVGREFVSGVALAPSGASYYKLAAIYQKGRRPLDG
APT73.201
(SEQ ID NO: 33)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGGREFVSGVALAPSGASYYKLAAIYQKGRRCLDG
APT73.202
(SEQ ID NO: 34)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEPGREFVSGVALAPSGASYYKLAAIYQKGRRCLDG
APT73.203
(SEQ ID NO: 35)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEVGREFVSGVALAPSGASYYKLAAIYQKGRRCLDG
APT73.204
(SEQ ID NO: 36)
MDEVYAAVEQTSRLLDVPCSPDRFEPVWKAFGDQLPDSVLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAAIYQKGRRPLDG
APT73 (Natural Aromatic Prentyltransferase)
(SEQ ID NO: 37)
MDEVYAAVEQTSRLLDVPCSPDRFEPVWKAFGDQLPDSHLVFSMAAGEAHRGELDFDFSL
RPEGADPYTTALEHGFIEPTDHPVGSVLAEVGKRFAIASYGVEYGVVGGFKKSYAFFPLD
DFPPLAQFAEVPSVPPCLAGHVETLTRLGFDDKVSAIGVNYRKNTLNVYLAASAVDTGDK
LALLRAFGYPEPDARVRQFIERSFSLYPTFNWDSSAAERICFSVKTQQPGELPAPHDEPT
EAFARQVPHVYEGGREFVSAVALAPSGASYYKLAAYYQKARGASNAAFAAKREDAAA
APT73.77 (Mutant Aromatic Prenyltransferase)
(SEQ ID NO: 38)
MDEVYAAVEQTSRLLDVPCSPDRFEPVWKAFGDQLPDSHLVFSMAAGEAHRGELDFDFSL
RPEGADPYTTALEHGFIEPTDHPVGSVLAEVGKRFAIASYGVEYGVVGGFKKSYAFFPLD
DFPPLAQFAEVPSVPPCLAGHVETLTRLGFDDKVSIIGVNYRKNTLNVYLAASAVDTGDK
LALLRAFGYPEPDARVRQFIERSFRLYPTFNWDSSAAERICFAVHTQQPGELPAPHDEPT
EAFARQVPHVYEGGREFVSGVALAPSGASYYKLAALYQKARRCLDG
APT73.248
(SEQ ID NO: 96)
MDEVYAAVEQTSRLLDVPCSPDRFEPVWKAFGDQLPDSHLLFSMAAGEAHRGELDFDFSL
RPEGADPYTTALEHGFIEPTDHPVGSVLAEVGKRFAIASYGVEYGVVGGFKKSYAFFPLD
DFPPLAEFARIPSVPPCLAGHVETLTRLGFDDKVSIIGVNYQKKTLNVYLAASAVDTGDK
LALLRAFGYPEPDARVRQFIERSFRLYPTFNWDSSAAERICFAVHTNQPGELPAPHDEPT
EAFARQVPHVYEGPREFVSGVALAPSGASYYKLAAIYQKGRRPLDA
MEMBRANE-BOUND PRENYLTRANSFERASE ENZYMES
MPT4.33
(SEQ ID NO: 39)
MSDNSIATKILNFGHTCWKLQRPYAVKGMISIACGLFGRELFNNRHLFSWGLMWKAFFAL
VPILSFNFFAAVMNQIYDVDIDRINKPDLPLVSGEMSIETAWILSIIVALTGLIVTIKLK
SAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYY
AEYFVYVFI
MPT4.29
(SEQ ID NO: 40)
MSDNSIATKILNFGHTCWKLQRPYAVKGIISIACGLFGRELFNNRHLFSWGLMWKAFFAL
VPILSFNFFAAIMNQIYDVDIDRINKPDLPLVSGEMSIETAWILSIIVALTGLIVTIKLK
SAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYY
AEYFVYVFI
MPT4.30
(SEQ ID NO: 41)
MSDNSIATKILNFGHTCWKLQRPYAVKGVISIACGLFGRELFNNRHLFSWGLMWKAFFAL
VPILSFNFFAAIMNQIYDVDIDRINKPDLPLVSGEMSIETAWILSIIVALTGLIVTIKLK
SAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYY
AEYFVYVFI
MPT4.32
(SEQ ID NO: 42)
MSDNSIATKILNFGHTCWKLQRPYAVKGMISIACGLFGRELFNNRHLFSWGLMWKAFFAL
VPILSFNFFAACMNQIYDVDIDRINKPDLPLVSGEMSIETAWILSIIVALTGLIVTIKLK
SAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYY
AEYFVYVFI
MPT4.34
(SEQ ID NO: 43)
MSDNSIATKILNFGHTCWKLQRPYAVKGMISIACGLFGRELFNNRHLFSWGLMWKAFFAL
VPILSFNFFAAIMNQIYDVDIDRINKPDLPLVSGEMSIETAWILSIIVALTGLIVTIKLK
SAPLFVFIYIFGIFMGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYY
AEYFVYVFI
MPT4.40
(SEQ ID NO: 44)
MSDNSIATKILNFGHTCWKLQRPYAVKGMISIACGLFGRELFNNRHLFSWGLMWKAFFAL
VPILSFNFFAAIMNQIYDVDIDRINKPDLPLVSGEMSIETAWILSIIVALTGLIVTIKLK
SAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQLFKSNIMILSHAILAFCLIFQTRELALANYASAPSRQFFEFIWLLYY
AEYFVYVFI
MPT4.41
(SEQ ID NO: 45)
MSDNSIATKILNFGHTCWKLQRPYAVKGMISIACGLFGRELFNNRHLFSWGLMWKAFFAL
VPILSFNFFAAAMNQIYDVDIDRINKPDLPLVSGEMSIETAWILSIIVALTGLIVTIKLK
SAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYY
AEYFVYVFI
MPT4.1
(SEQ ID NO: 46)
MSDNSIATKILNFGHTCWKLQRPYAVKGMISIACGLFGRELFNNRHLFSWGLMWKAFFAL
VPILSFNFFAAIMNQIYDVDIDRINKPDLPLVSGEMSIETAWILSIIVALTGLIVTIKLK
SAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYY
AEYFVYVFI
MPT48
(SEQ ID NO: 47)
MPATRTPIHPEAAAYKNPRYQSGPLSVIPKSFVPYCELMRLELPHGNFLGYFPHLVGLLY
GSSASPARLPANDVVFQAALYIGWTFFMRGAGCAWNDVVDQDFDRKTTRCRVRPVARGAV
STTGANIFALAMVSLAFACISPLPAECQRLGLITTVLSIIYPFCKRVTNFAQVILGMTLA
INFILAAYGAGLPAVEAPYTLPTICVTTAITLLVVFYDVVYARQDTADDLKSGVKGMAVL
FRNYVEILLTSITLVIAGLLATTGVLVDNGPYFFVESVAGLLAALLAMIGGIRYRIFHTW
NSYSGWFYALAIFNLLGGYLIEYLDQVPMLNKA
MPT69
(SEQ ID NO: 48)
MSAKVASIPYTNPRYESGPLSAIPKSWVPYFELMRFELPHGYYLGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIVTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYSG
WFYVIAIINLMSGYFIEYLGNAPLLARAS
MPT69.2
(SEQ ID NO: 98)
MSAKVASIPYTNPRYESGPLSAIPKSWVPYFELMRFELPHGYYLGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIFTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYSG
WFYVIAIINLMSGYFIEYLGNAPLLARAS
MPT69.5
(SEQ ID NO: 99)
MSAKVASIPYTNPRYESGPLSAIPKSWVPYFELMRFELPHGYYLGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIHTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYSG
WFYVIAIINLMSGYFIEYLGNAPLLARAS
MPT69.6
(SEQ ID NO: 100)
MSAKVASIPYTNPRYERGPLSAIPKSWVPYFELMRFELPHGYYLGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIVTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYSG
WFYVIAIINLMSGYFIEYLGNAPLLARAS
MPT69.7
(SEQ ID NO: 101)
MSAKVASIPYTNPRYESGPLSAIPKSWVPYFELMRFELPHGYYLGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIVTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYPG
WFYVIAIINLMSGYFIEYLGNAPLLARAS
MPT69.8
(SEQ ID NO: 102)
MSAKVASIPYTNPRYESGPLSAIPKSWVPYFELMRFELPHGYYLGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIVTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYTG
WFYVIAIINLMSGYFIEYLGNAPLLARAS
MPT69.9
(SEQ ID NO: 103)
MSAKVASIPYTNPRYESGPLSAIPKSWVPYFELMRFELPHGYYLGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIVTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYWG
WFYVIAIINLMSGYFIEYLGNAPLLARAS
MPT69.10
(SEQ ID NO: 104)
MSAKVASIPYTNPRYESGPLSAIPKSWVPYFELMRFELPHGYYIGYFPHLVGIMYGASAG
PERLPASQLLFQALLYVGWTFCMRGAGCAWNDNIDQDFDRKTERCRTRPIARGAVSTTAG
HIFAISFVAAAFLCLAPLPTECHQLGVIVTVLSIIYPFCKRFTNFAQVILGFTLAANFIL
AAYGAGLPALEQPYTRPTACVTLAITLLVVFYDVVYARQDTADDLKSGVKGMAVLFRNHI
EILLAGLTCTIGGLLAVTGIYVENGPYYFVFSVAGLTVALLAMIGGIRYRVFHSWNSYSG
WFYVIAIINLMSGYFIEYLGNAPLLARAS
GERANYL DIPHOSPHATE SYNTHASE PROTEINS
GPS1.1
(SEQ ID NO: 49)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQK
LINKER PEPTIDES
L11 linker
(SEQ ID NO: 50)
GGAEAAAKEAAAKAGGSGGGSGGGGSGGSGGGGSGGGGS
L18 linker
(SEQ ID NO: 51)
GGGGSLEDPAVWEAGKVVAKGVGTADITATTSNGLIASSEEAD
NAATS
GERANYL PYROPHOSPHATE SYNTHASE/SOLUBLE AROMATIC
PRENYLTRANSFERASE FUSION PROTEINS
GPS1.1-L18-APT73.119
(SEQ ID NO: 52)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAALYQKGRRCLDG
GPS1.1-L18-APT73.159
(SEQ ID NO: 53)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTNQPGELPAPHDEPTEAFARQVPHVYEGGREFVSGVALAPSGA
SYYKLAALYQKGRRCLDG
GPS1.1-L18-APT73.160
(SEQ ID NO: 54)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGPREFVSGVALAPSGA
SYYKLAALYQKGRRCLDG
GPS1.1-L18-APT73.161
(SEQ ID NO: 55)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGVREFVSGVALAPSGA
SYYKLAALYQKGRRCLDG
GPS1.1-L18-APT73.162
(SEQ ID NO: 56)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGSREFVSGVALAPSGA
SYYKLAALYQKGRRCLDG
GPS1.1-L18-APT73.163
(SEQ ID NO: 57)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAAIYQKGRRCLDG
GPS1.1-L18-APT73.165
(SEQ ID NO: 58)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAAIYQKGRRCLDG
GPS1.1-L18-APT73.166
(SEQ ID NO: 59)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAALYQKGRKCLDG
GPS1.1-L18-APT73.168
(SEQ ID NO: 60)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAALYQKGRRELDG
GPS1.1-L18-APT73.169
(SEQ ID NO: 61)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAALYQKGRRPLDG
GPS1.1-L18-APT73.170
(SEQ ID NO: 62)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAALYQKGRRCLDGRYG
GPS1.1-L18-APT73.172
(SEQ ID NO: 63)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAALYQKGRRCLDGHRR
GPS1.1-L18-APT73.179
(SEQ ID NO: 64)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLCFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKNTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAALYQKGRRCLDG
GPS1.1-L18-APT73.182
(SEQ ID NO: 65)
MSKAKFESVFPRISEELVQLLRDEGLPQDAVQWESDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGPREFVSGVALAPSGA
SYYKLAAIYQKGRRCLDG
GPS1.1-L18-APT73.183
(SEQ ID NO: 66)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEGPREFVSGVALAPSGA
SYYKLAAIYQKGRRPLDG
GPS1.1-L18-APT73.184
(SEQ ID NO: 67)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGVREFVSGVALAPSGA
SYYKLAAIYQKGRRCLDG
GPS1.1-L18-APT73.185
(SEQ ID NO: 68)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEGVREFVSGVALAPSGA
SYYKLAAIYQKGRRPLDG
GPS1.1-L18-APT73.186
(SEQ ID NO: 69)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGGREFVSGVALAPSGA
SYYKLAAIYQKGRRPLDG
GPS1.1-L18-APT73.187
(SEQ ID NO: 70)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGPREFVSGVALAPSGA
SYYKLAAIYQKGRRPLDG
GPS1.1-L18-APT73.188
(SEQ ID NO: 71)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGVREFVSGVALAPSGA
SYYKLAAIYQKGRRPLDG
GPS1.1-L18-APT73.189
(SEQ ID NO: 72)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGGREFVSGVALAPSGA
SYYKLAAIYQKGRRCLDG
GPS1.1-L18-APT73.190
(SEQ ID NO: 73)
MSKAKFESVFPRISEELVQLLRDEGLPQDAVQWESDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEPGREFVSGVALAPSGA
SYYKLAAIYQKGRRCLDG
GPS1.1-L18-APT73.191
(SEQ ID NO: 74)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEVGREFVSGVALAPSGA
SYYKLAAIYQKGRRCLDG
GPS1.1-L18-APT73.192
(SEQ ID NO: 75)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAAIYQKGRRPLDG
GPS1.1-L18-APT73.193
(SEQ ID NO: 76)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAAIYQKGRRCLDG
GPS1.1-L18-APT73.194
(SEQ ID NO: 77)
MSKAKFESVFPRISEELVQLLRDEGLPQDAVQWESDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEPGREFVSGVALAPSGA
SYYKLAAIYQKGRRCLDG
GPS1.1-L18-APT73.195
(SEQ ID NO: 78)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEPGREFVSGVALAPSGA
SYYKLAAIYQKGRRPLDG
GPS1.1-L18-APT73.196
(SEQ ID NO: 79)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEVGREFVSGVALAPSGA
SYYKLAAIYQKGRRCLDG
GPS1.1-L18-APT73.197
(SEQ ID NO: 80)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEVGREFVSGVALAPSGA
SYYKLAAIYQKGRRPLDG
GPS1.1-L18-APT73.198
(SEQ ID NO: 81)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGGREFVSGVALAPSGA
SYYKLAAIYQKGRRPLDG
GPS1.1-L18-APT73.199
(SEQ ID NO: 82)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEPGREFVSGVALAPSGA
SYYKLAAIYQKGRRPLDG
GPS1.1-L18-APT73.200
(SEQ ID NO: 83)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEVGREFVSGVALAPSGA
SYYKLAAIYQKGRRPLDG
GPS1.1-L18-APT73.201
(SEQ ID NO: 84)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGGREFVSGVALAPSGA
SYYKLAAIYQKGRRCLDG
GPS1.1-L18-APT73.202
(SEQ ID NO: 85)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEPGREFVSGVALAPSGA
SYYKLAAIYQKGRRCLDG
GPS1.1-L18-APT73.203
(SEQ ID NO: 86)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEVGREFVSGVALAPSGA
SYYKLAAIYQKGRRCLDG
GPS1.1-L18-APT73.204
(SEQ ID NO: 87)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSVLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTQQPGELPAPHDEPTEAFARQVPHVYEGGREFVSGVALAPSGA
SYYKLAAIYQKGRRPLDG
GPS1.1-L18-APT73.248
(SEQ ID NO: 97)
MSKAKFESVFPRISEELVQLLRDEGLPQDAVQWFSDSLQYNCVGGKLNRGLSVVDTYQLL
TGKKELDDEEYYRLALLGWLIELLQAFWLVSDDIMDESKTRRGQPCWYLKPKVGMIAIWD
AFMLESGIYILLKKHFRQEKYYIDLVELFHDISFKTELGQLVDLLTAPEDEVDLNRFSLD
KHSFIVRYKTAYYSFYLPVVLAMYVAGITNPKDLQQAMDVLIPLGEYFQVQDDYLDNFGD
PEFIGKIGTDIQDNKCSWLVNKALQKATPEQRQILEDNYGVKDKSKELVIKKLYDDMKIE
QDYLDYEEEVVGDIKKKIEQVDESRGFKKEVLNAFLAKIYKRQKGGGGSLEDPAVWEAGK
VVAKGVGTADITATTSNGLIASSEEADNAATSMDEVYAAVEQTSRLLDVPCSPDRFEPVW
KAFGDQLPDSHLLFSMAAGEAHRGELDFDFSLRPEGADPYTTALEHGFIEPTDHPVGSVL
AEVGKRFAIASYGVEYGVVGGFKKSYAFFPLDDFPPLAEFARIPSVPPCLAGHVETLTRL
GFDDKVSIIGVNYQKKTLNVYLAASAVDTGDKLALLRAFGYPEPDARVRQFIERSFRLYP
TFNWDSSAAERICFAVHTNQPGELPAPHDEPTEAFARQVPHVYEGPREFVSGVALAPSGA
SYYKLAAIYQKGRRPLDA
GERANYL PYROPHOSPHATE SYNTHASE/MEMBRANE-BOUND
PRENYLTRANSFERASE FUSION PROTEINS
GPS1.1-L11-MPT4.1
(SEQ ID NO: 88)
SKAKFESVFPRISEELVQLLRDEGLPQDAVQWFSDSLQYNCVGGKLNRGLSVVDTYQLLT
GKKELDDEEYYRLALLGWLIELLQAFWLVSDDIMDESKTRRGQPCWYLKPKVGMIAIWDA
FMLESGIYILLKKHFRQEKYYIDLVELFHDISFKTELGQLVDLLTAPEDEVDLNRFSLDK
HSFIVRYKTAYYSFYLPVVLAMYVAGITNPKDLQQAMDVLIPLGEYFQVQDDYLDNFGDP
EFIGKIGTDIQDNKCSWLVNKALQKATPEQRQILEDNYGVKDKSKELVIKKLYDDMKIEQ
DYLDYEEEVVGDIKKKIEQVDESRGFKKEVLNAFLAKIYKRQKGGAEAAAKEAAAKAGGS
GGGSGGGGSGGSGGGGSGGGGSMSDNSIATKILNFGHTCWKLQRPYAVKGMISIACGLFG
RELFNNRHLFSWGLMWKAFFALVPILSFNFFAAIMNQIYDVDIDRINKPDLPLVSGEMSI
ETAWILSIIVALTGLIVTIKLKSAPLFVFIYIFGIFAGFAYSVPPIRWKQYPFTNFLITI
SSHVGLAFTSYSATTSALGLPFVWRPAFSFIIAFMTVMGMTIAFAKDISDIEGDAKYGVS
TVATKLGARNMTFVVSGVLLLNYLVSISIGIIWPQVFKSNIMILSHAILAFCLIFQTREL
ALANYASAPSRQFFEFIWLLYYAEYFVYVFI
GPS1.1-L11-MPT4.29
(SEQ ID NO: 89)
SKAKFESVFPRISEELVQLLRDEGLPQDAVQWFSDSLQYNCVGGKLNRGLSVVDTYQLLT
GKKELDDEEYYRLALLGWLIELLQAFWLVSDDIMDESKTRRGQPCWYLKPKVGMIAIWDA
FMLESGIYILLKKHFRQEKYYIDLVELFHDISFKTELGQLVDLLTAPEDEVDLNRFSLDK
HSFIVRYKTAYYSFYLPVVLAMYVAGITNPKDLQQAMDVLIPLGEYFQVQDDYLDNFGDP
EFIGKIGTDIQDNKCSWLVNKALQKATPEQRQILEDNYGVKDKSKELVIKKLYDDMKIEQ
DYLDYEEEVVGDIKKKIEQVDESRGFKKEVLNAFLAKIYKRQKGGAEAAAKEAAAKAGGS
GGGSGGGGSGGSGGGGSGGGGSMSDNSIATKILNFGHTCWKLQRPYAVKGIISIACGLFG
RELFNNRHLFSWGLMWKAFFALVPILSFNFFAAIMNQIYDVDIDRINKPDLPLVSGEMSI
ETAWILSIIVALTGLIVTIKLKSAPLFVFIYIFGIFAGFAYSVPPIRWKQYPFTNFLITI
SSHVGLAFTSYSATTSALGLPFVWRPAFSFIIAFMTVMGMTIAFAKDISDIEGDAKYGVS
TVATKLGARNMTFVVSGVLLLNYLVSISIGIIWPQVFKSNIMILSHAILAFCLIFQTREL
ALANYASAPSRQFFEFIWLLYYAEYFVYVFI
GPS1.1-L11-MPT4.30
(SEQ ID NO: 90)
SKAKFESVFPRISEELVQLLRDEGLPQDAVQWFSDSLQYNCVGGKLNRGLSVVDTYQLLT
GKKELDDEEYYRLALLGWLIELLQAFWLVSDDIMDESKTRRGQPCWYLKPKVGMIAIWDA
FMLESGIYILLKKHFRQEKYYIDLVELFHDISFKTELGQLVDLLTAPEDEVDLNRFSLDK
HSFIVRYKTAYYSFYLPVVLAMYVAGITNPKDLQQAMDVLIPLGEYFQVQDDYLDNFGDP
EFIGKIGTDIQDNKCSWLVNKALQKATPEQRQILEDNYGVKDKSKELVIKKLYDDMKIEQ
DYLDYEEEVVGDIKKKIEQVDESRGFKKEVLNAFLAKIYKRQKGGAEAAAKEAAAKAGGS
GGGSGGGGSGGSGGGGSGGGGSMSDNSIATKILNFGHTCWKLQRPYAVKGVISIACGLFG
RELFNNRHLFSWGLMWKAFFALVPILSFNFFAAIMNQIYDVDIDRINKPDLPLVSGEMSI
ETAWILSIIVALTGLIVTIKLKSAPLFVFIYIFGIFAGFAYSVPPIRWKQYPFTNFLITI
SSHVGLAFTSYSATTSALGLPFVWRPAFSFIIAFMTVMGMTIAFAKDISDIEGDAKYGVS
TVATKLGARNMTFVVSGVLLLNYLVSISIGIIWPQVFKSNIMILSHAILAFCLIFQTREL
ALANYASAPSRQFFEFIWLLYYAEYFVYVFI
GPS1.1-L11-MPT4.32
(SEQ ID NO: 91)
SKAKFESVFPRISEELVQLLRDEGLPQDAVQWESDSLQYNCVGGKLNRGLSVVDTYQLLT
GKKELDDEEYYRLALLGWLIELLQAFWLVSDDIMDESKTRRGQPCWYLKPKVGMIAIWDA
FMLESGIYILLKKHFRQEKYYIDLVELFHDISFKTELGQLVDLLTAPEDEVDLNRFSLDK
HSFIVRYKTAYYSFYLPVVLAMYVAGITNPKDLQQAMDVLIPLGEYFQVQDDYLDNFGDP
EFIGKIGTDIQDNKCSWLVNKALQKATPEQRQILEDNYGVKDKSKELVIKKLYDDMKIEQ
DYLDYEEEVVGDIKKKIEQVDESRGFKKEVLNAFLAKIYKRQKGGAEAAAKEAAAKAGGS
GGGSGGGGSGGSGGGGSGGGGSMSDNSIATKILNFGHTCWKLQRPYAVKGMISIACGLFG
RELFNNRHLFSWGLMWKAFFALVPILSENFFAACMNQIYDVDIDRINKPDLPLVSGEMSI
ETAWILSIIVALTGLIVTIKLKSAPLFVFIYIFGIFAGFAYSVPPIRWKQYPFTNFLITI
SSHVGLAFTSYSATTSALGLPFVWRPAFSFIIAFMTVMGMTIAFAKDISDIEGDAKYGVS
TVATKLGARNMTFVVSGVLLLNYLVSISIGIIWPQVFKSNIMILSHAILAFCLIFQTREL
ALANYASAPSRQFFEFIWLLYYAEYFVYVFI
GPS1.1-L11-MPT4.33
(SEQ ID NO: 92)
SKAKFESVFPRISEELVQLLRDEGLPQDAVQWFSDSLQYNCVGGKLNRGLSVVDTYQLLT
GKKELDDEEYYRLALLGWLIELLQAFWLVSDDIMDESKTRRGQPCWYLKPKVGMIAIWDA
FMLESGIYILLKKHFRQEKYYIDLVELFHDISFKTELGQLVDLLTAPEDEVDLNRFSLDK
HSFIVRYKTAYYSFYLPVVLAMYVAGITNPKDLQQAMDVLIPLGEYFQVQDDYLDNFGDP
EFIGKIGTDIQDNKCSWLVNKALQKATPEQRQILEDNYGVKDKSKELVIKKLYDDMKIEQ
DYLDYEEEVVGDIKKKIEQVDESRGFKKEVLNAFLAKIYKRQKGGAEAAAKEAAAKAGGS
GGGSGGGGSGGSGGGGSGGGGSMSDNSIATKILNFGHTCWKLQRPYAVKGMISIACGLFG
RELFNNRHLFSWGLMWKAFFALVPILSFNFFAAVMNQIYDVDIDRINKPDLPLVSGEMSI
ETAWILSIIVALTGLIVTIKLKSAPLFVFIYIFGIFAGFAYSVPPIRWKQYPFTNFLITI
SSHVGLAFTSYSATTSALGLPFVWRPAFSFIIAFMTVMGMTIAFAKDISDIEGDAKYGVS
TVATKLGARNMTFVVSGVLLLNYLVSISIGIIWPQVFKSNIMILSHAILAFCLIFQTREL
ALANYASAPSRQFFEFIWLLYYAEYFVYVFI
GPS1.1-L11-MPT4.34
(SEQ ID NO: 93)
SKAKFESVFPRISEELVQLLRDEGLPQDAVQWFSDSLQYNCVG
GKLNRGLSVVDTYQLLTGKKELDDEEYYRLALLGWLIELLQAFWLVSDDIMDESKTRRGQ
PCWYLKPKVGMIAIWDAFMLESGIYILLKKHFRQEKYYIDLVELFHDISFKTELGQLVDL
LTAPEDEVDLNRFSLDKHSFIVRYKTAYYSFYLPVVLAMYVAGITNPKDLQQAMDVLIPL
GEYFQVQDDYLDNFGDPEFIGKIGTDIQDNKCSWLVNKALQKATPEQRQILEDNYGVKDK
SKELVIKKLYDDMKIEQDYLDYEEEVVGDIKKKIEQVDESRGFKKEVLNAFLAKIYKRQK
GGAEAAAKEAAAKAGGSGGGSGGGGSGGSGGGGSGGGGSMSDNSIATKILNFGHTCWKLQ
RPYAVKGMISIACGLFGRELFNNRHLFSWGLMWKAFFALVPILSFNFFAAIMNQIYDVDI
DRINKPDLPLVSGEMSIETAWILSIIVALTGLIVTIKLKSAPLFVFIYIFGIFMGFAYSV
PPIRWKQYPFTNFLITISSHVGLAFTSYSATTSALGLPFVWRPAFSFIIAFMTVMGMTIA
FAKDISDIEGDAKYGVSTVATKLGARNMTFVVSGVLLLNYLVSISIGIIWPQVFKSNIMI
LSHAILAFCLIFQTRELALANYASAPSRQFFEFIWLLYYAEYFVYVFI
GPS1.1-L11-MPT4.40
(SEQ ID NO: 94)
SKAKFESVFPRISEELVQLLRDEGLPQDAVQWESDSLQYNCVGGKLNRGLSVVDTYQLLT
GKKELDDEEYYRLALLGWLIELLQAFWLVSDDIMDESKTRRGQPCWYLKPKVGMIAIWDA
FMLESGIYILLKKHFRQEKYYIDLVELFHDISFKTELGQLVDLLTAPEDEVDLNRFSLDK
HSFIVRYKTAYYSFYLPVVLAMYVAGITNPKDLQQAMDVLIPLGEYFQVQDDYLDNFGDP
EFIGKIGTDIQDNKCSWLVNKALQKATPEQRQILEDNYGVKDKSKELVIKKLYDDMKIEQ
DYLDYEEEVVGDIKKKIEQVDESRGFKKEVLNAFLAKIYKRQKGGAEAAAKEAAAKAGGS
GGGSGGGGSGGSGGGGSGGGGSMSDNSIATKILNFGHTCWKLQRPYAVKGMISIACGLFG
RELFNNRHLFSWGLMWKAFFALVPILSFNFFAAIMNQIYDVDIDRINKPDLPLVSGEMSI
ETAWILSIIVALTGLIVTIKLKSAPLFVFIYIFGIFAGFAYSVPPIRWKQYPFTNFLITI
SSHVGLAFTSYSATTSALGLPFVWRPAFSFIIAFMTVMGMTIAFAKDISDIEGDAKYGVS
TVATKLGARNMTFVVSGVLLLNYLVSISIGIIWPQLFKSNIMILSHAILAFCLIFQTREL
ALANYASAPSRQFFEFIWLLYYAEYFVYVFI
GPS1.1-L11-MPT4.41
(SEQ ID NO: 95)
SKAKFESVFPRISEELVQLLRDEGLPQDAVQWFSDSLQYNCVGGKLNRGLSVVDTYQLLT
GKKELDDEEYYRLALLGWLIELLQAFWLVSDDIMDESKTRRGQPCWYLKPKVGMIAIWDA
FMLESGIYILLKKHFRQEKYYIDLVELFHDISFKTELGQLVDLLTAPEDEVDLNRFSLDK
HSFIVRYKTAYYSFYLPVVLAMYVAGITNPKDLQQAMDVLIPLGEYFQVQDDYLDNFGDP
EFIGKIGTDIQDNKCSWLVNKALQKATPEQRQILEDNYGVKDKSKELVIKKLYDDMKIEQ
DYLDYEEEVVGDIKKKIEQVDESRGFKKEVLNAFLAKIYKRQKGGAEAAAKEAAAKAGGS
GGGSGGGGSGGSGGGGSGGGGSMSDNSIATKILNFGHTCWKLQRPYAVKGMISIACGLFG
RELFNNRHLFSWGLMWKAFFALVPILSFNFFAAAMNQIYDVDIDRINKPDLPLVSGEMSI
ETAWILSIIVALTGLIVTIKLKSAPLFVFIYIFGIFAGFAYSVPPIRWKQYPFTNFLITI
SSHVGLAFTSYSATTSALGLPFVWRPAFSFIIAFMTVMGMTIAFAKDISDIEGDAKYGVS
TVATKLGARNMTFVVSGVLLLNYLVSISIGIIWPQVFKSNIMILSHAILAFCLIFQTREL
ALANYASAPSRQFFEFIWLLYYAEYFVYVFI
FARNESYL DIPHOSPHATE SYNTHASE
GPS1.A28
(SEQ ID NO: 105)
MSKAKFESVFPRISEELVQLLRDEGLPQDAVQWFSDSLQYNCVGGKLNRGLSVVDTYQLL
TGKKELDDEEYYRLALLGWLIELLQAFLSDDIMDESKTRRGQPCWYLKPKVGMIAINDAF
MLESGIYILLKKHFRQEKYYIDLVELFHDISFKTELGQLVDLLTAPEDEVDLNRFSLDKH
SFIVRYKTAYYSFYLPVVLAMYVAGITNPKDLQQAMDVLIPLGEYFQVQDDYLDNFGDPE
FIGKIGTDIQDNKCSWLVNKALQKATPEQRQILEDNYGVKDKSKELVIKKLYDDMKIEQD
YLDYEEEVVGDIKKKIEQVDESRGFKKEVLNAFLAKIYKRQK*

1.-46. (canceled)

47. An engineered prenyltransferase selected from the group consisting of:
a) an aromatic prenyltransferase (APT) engineered to transfer geranyl pyrophosphate (GPP) to olivetolic acid and/or divarinic acid with an at least two times higher efficiency compared to a mutant aromatic prenyltransferase APT73.77 (SEQ ID NO: 38); and
b) a recombinant membrane-bound prenyltransferase (rMPT), said rMPT engineered to transfer geranyl pyrophosphate (GPP) to olivetolic acid and/or divarinic acid with higher efficiency compared to a naturally occurring MPT, wherein said rMPT has an amino acid sequence comprising at least one amino acid modification compared to the amino acid sequence of MPT69 (SEQ ID NO: 48), MPT4.1 (SEQ ID NO: 46), or MPT48 (SEQ ID NO: 47)

48. The engineered prenyltransferase of claim 47, wherein said engineered APT contains an amino acid sequence with at least two amino acid modifications compared to SEQ ID NO: 38, wherein a first of the at least two amino acid modifications corresponds to a substitution, deletion or insertion at amino acid position R162 of SEQ ID NO: 38, and/or wherein the second of the at least two amino acid modifications compared to the amino acid sequence APT73.77 is selected from a substitution, deletion or insertion at an amino acid position corresponding to position H39, V41, Q127, E130, V131, 1156, N164, R205, Δ223, H225, Q227, G254, G260, L276, G280, R282, C283, L284, D285, or G286 of SEQ ID NO: 38.

49. The engineered prenyltransferase of claim 47, wherein the at least two amino acid modifications comprise a second, third, fourth, and fifth or more amino acid modifications, selected from a substitution, deletion or insertion at four or more amino acid positions corresponding to positions H39, V41, Q127, E130, V131, 1156, N164, R205, Δ223, H225, Q227, G254, G260, L276, G280, R282, C283, L284, D285, and/or G286 of SEQ ID NO: 38, wherein the modifications do not comprise the substitution R205S.

50. The engineered prenyltransferase of claim 47, wherein the engineered APT comprises an amino acid sequence with at least 90% identity to the amino acid sequence of APT73.179 (SEQ ID NO: 1), APT73.119 (SEQ ID NO: 2), APT73.159 (SEQ ID NO: 3), APT73.160 (SEQ ID NO: 4), APT73.161 (SEQ ID NO: 5), APT73.162 (SEQ ID NO: 6), APT73.163 (SEQ ID NO: 7), APT73.165 (SEQ ID NO: 8), APT73.166 (SEQ ID NO: 9), APT73.168 (SEQ ID NO: 10), APT73.169 (SEQ ID NO: 11), APT73.170 (SEQ ID NO: 12), APT73.172 (SEQ ID NO: 13), APT73.182 (SEQ ID NO: 14), APT73.183 (SEQ ID NO: 15), APT73.184 (SEQ ID NO: 16), APT73.185 (SEQ ID NO: 17), APT73.186 (SEQ ID NO: 18), APT73.187 (SEQ ID NO: 19), APT73.188 (SEQ ID NO: 20), APT73.73.189 (SEQ ID NO:21), APT73.190 (SEQ ID NO: 22), APT73.191 (SEQ ID NO: 23), APT73.192 (SEQ ID NO: 24), APT73.193 (SEQ ID NO: 25), APT73.194 (SEQ ID NO: 26), APT73.195 (SEQ ID NO: 27), APT73.196 (SEQ ID NO: 28), APT73.197 (SEQ ID NO: 29), APT73.199 (SEQ ID NO: 31), APT73.200 (SEQ ID NO: 32), APT73.201 (SEQ ID NO: 33), APT73.202 (SEQ ID NO: 34), APT73.203 (SEQ ID NO: 35), APT73.204 (SEQ ID NO: 36), APT73.248 (SEQ ID NO: 96) or a functional fragment or variant thereof.

51. The engineered prenyltransferase of claim 50, wherein the engineered APT comprises an amino acid sequence with at least 90% identity to the amino acid sequence of APT73.187 (SEQ ID NO: 96) or APT73.248 (SEQ ID NO: 96).

52. The engineered prenyltransferase of claim 48, wherein the at least two amino acid modifications comprise three or more amino acid modifications comprising the substitution R162Q, one or more substitutions selected from the group consisting of Q127E, E130R, V131I, and Δ280G, and one or more substitutions selected from the group consisting of H39V, V41C, and V41L, all as relating to the amino acid sequence of APT73.77 (SEQ ID NO: 38).

53. The engineered prenyltransferase of claim 48, wherein the engineered APT comprises an amino acid sequence with at least one amino acid modification as compared to APT73.179 (SEQ ID NO: 1), APT73.119 (SEQ ID NO: 2), APT73.159 (SEQ ID NO: 3), APT73.160 (SEQ ID NO: 4), APT73.161 (SEQ ID NO: 5), APT73.162 (SEQ ID NO: 6), APT73.163 (SEQ ID NO: 7), APT73.165 (SEQ ID NO: 8), APT73.166 (SEQ ID NO: 9), APT73.168 (SEQ ID NO: 10), APT73.169 (SEQ ID NO: 11), APT73.170 (SEQ ID NO: 12), APT73.172 (SEQ ID NO: 13), APT73.182 (SEQ ID NO: 14), APT73.183 (SEQ ID NO: 15), APT73.184 (SEQ ID NO: 16), APT73.185 (SEQ ID NO: 17), APT73.186 (SEQ ID NO: 18), APT73.187 (SEQ ID NO: 19), APT73.188 (SEQ ID NO: 20), APT73.189 (SEQ ID NO: 21), APT73.190 (SEQ ID NO: 22), APT73.191 (SEQ ID NO: 23), APT73.192 (SEQ ID NO: 24), APT73.193 (SEQ ID NO: 25), APT73.194 (SEQ ID NO: 26), APT73.195 (SEQ ID NO: 27), APT73.196 (SEQ ID NO: 28), APT73.197 (SEQ ID NO: 29), APT73.199 (SEQ ID NO: 31), APT73.200 (SEQ ID NO: 32), APT73.201 (SEQ ID NO: 33), APT73.202 (SEQ ID NO: 34), APT73.203 (SEQ ID NO: 35), APT73.204 (SEQ ID NO: 36), APT73.248 (SEQ ID NO: 96) or a functional fragment or variant thereof.

54. The engineered prenyltransferase of claim 47, wherein the rMPT comprises an amino acid sequence having at least one amino acid modification as compared to SEQ ID NO: 48 (MPT69) producing a mutant with one or both of i) increased activity for prenylation of OA and DVA to form CBGA and CBGVA, respectively, and ii) increased selectivity for GPP over FPP.

55. The engineered prenyltransferase of claim 54, wherein the at least one amino acid modification as compared to MPT69 (SEQ ID NO: 48) comprises one or more deletion, insertion or substitution at one or more amino acid positions corresponding to the first 45 amino acids of the N-terminus M1-G45, and/or H49, M83, R84, C88, Δ89, N91, D92, N93, 194, D95, Q96, D97, F98, D99, R104, R108, S116, Δ131, C142, H143, V149, K160, Q167, L170, T173, L174, Δ176, R196, T202, V209, Y212, D213, V214, Y216, T248, C249, V257, V262, N264, P266, L276, Δ279, Δ282, S299, G300, W301, N309, M311, S312, G313, Δ322, L324, and/or L325 of SEQ ID NO: 48.

56. The engineered prenyltransferase of claim 54, wherein said rMPT comprises an amino acid sequence with at least 90% identity to the amino acid sequence of MPT69.2 (SEQ ID NO: 98), MPT69.5 (SEQ ID NO: 99), MPT69.6 (SEQ ID NO: 100), MPT69.7 (SEQ ID NO: 101), MPT69.8 (SEQ ID NO: 102), MPT69.9 (SEQ ID NO: 103), or MPT69.10 (SEQ ID NO: 104).

57. A cell comprising the engineered prenyltransferase of claim 47, wherein the cell is:
i) capable of producing CBGA in the presence of GPP and OA;
ii) capable of producing CBGVA in the presence of GPP and DVA;
iii) is capable of making a cannabinoid in the presence of a carbon source and optionally, hexanoic or butyric acid;
iv) expresses an exogenous membrane transporter that improves OA or DVA uptake, optionally, wherein one or more genes in the cell encoding a protein that exports OA or DVA is down-regulated or deactivated; and/or
v) encodes an exogenous hexanoyl-CoA synthetase and/or butyryl-CoA synthetase.

58. The cell of claim 57, wherein the cell is a yeast cell or a bacterial cell, optionally, wherein the yeast cell is a Yarrowia strain or a Saccharomyces strain.

59. A method of producing CBGA, CBGVA or derivatives thereof comprising culturing the cell of claim 57 under suitable conditions to produce CBGA, CBGVA or derivatives thereof, optionally, wherein the suitable condition comprises supplementing a culture media in which the cell is cultured with at least one of butyric acid, valeric acid, isovaleric acid, hexanoic acid, hexanol, butanol, oleic acid, glycerol or glucose.

60. A fusion protein comprising a polypeptide having Geranyl diphosphate synthase (GPS) activity fused directly or indirectly to a polypeptide having prenyltransferase activity, wherein the polypeptide having prenyltransferase activity comprises:
a) a polypeptide sequence having at least 85% identity to the polypeptide sequence of MPT69 (SEQ ID NO: 48) or MPT48 (SEQ ID NO: 47),
b) a polypeptide sequence comprising at least one amino acid modification compared to the amino acid sequence of MPT4.1 (SEQ ID NO: 46), or
c) a polypeptide comprising an amino acid sequence with at least two amino acid modifications compared to SEQ ID NO: 38, wherein a first of the at least two amino acid modifications corresponds to a substitution, deletion or insertion at amino acid position R162 of SEQ ID NO: 38, and wherein the N-terminal of the polypeptide having prenyltransferase activity is fused to the C-terminal of the GPS, or the C-terminal of the polypeptide having prenyltransferase activity is fused to the N-terminal of the GPS.

61. The fusion protein of claim 60, wherein the fusion protein comprises a polypeptide having at least 90% identity to GPS1.1-L18-APT73.119 (SEQ ID NO: 52), GPS1.1-L18-APT73.159 (SEQ ID NO: 53), GPS1.1-L18-APT73.160 (SEQ ID NO: 54), GPS1.1-L18-APT73.161 (SEQ ID NO: 55), GPS1.1-L18-APT73.162 (SEQ ID NO: 56), GPS1.1-L18-APT73.163 (SEQ ID NO: 57), GPS1.1-L18-APT73.165 (SEQ ID NO: 58), GPS1.1-L18-APT73.166 (SEQ ID NO: 59), GPS1.1-L18-APT73.168 (SEQ ID NO: 60), GPS1.1-L18-APT73.169 (SEQ ID NO: 61), GPS1.1-L18-APT73.170 (SEQ ID NO: 62), GPS1.1-L18-APT73.172 (SEQ ID NO: 63), GPS1.1-L18-APT73.179 (SEQ ID NO: 64), GPS1.1-L18-APT73.182 (SEQ ID NO: 65), GPS1.1-L18-APT73.183 (SEQ ID NO: 66), GPS1.1-L18-APT73.184 (SEQ ID NO: 67), GPS1.1-L18-APT73.185 (SEQ ID NO: 68), GPS1.1-L18-APT73.186 (SEQ ID NO: 69), GPS1.1-L18-APT73.187 (SEQ ID NO: 70), GPS1.1-L18-APT73.188 (SEQ ID NO: 71), GPS1.1-L18-APT73.189 (SEQ ID NO: 72), GPS1.1-L18-APT73.190 (SEQ ID NO: 73), GPS1.1-L18-APT73.191 (SEQ ID NO: 74), GPS1.1-L18-APT73.192 (SEQ ID NO: 75), GPS1.1-L18-APT73.193 (SEQ ID NO: 76), GPS1.1-L18-APT73.194 (SEQ ID NO: 77), GPS1.1-L18-APT73.195 (SEQ ID NO: 78), GPS1.1-L18-APT73.196 (SEQ ID NO: 79), GPS1.1-L18-APT73.197 (SEQ ID NO: 80), GPS1.1-L18-APT73.199 (SEQ ID NO: 82), GPS1.1-L18-APT73.200 (SEQ ID NO: 83), GPS1.1-L18-APT73.201 (SEQ ID NO: 84), GPS1.1-L18-APT73.202 (SEQ ID NO: 85), GPS1.1-L18-APT73.203 (SEQ ID NO: 86), GPS1.1-L18-APT73.204 (SEQ ID NO: 87), or GPS1.1-L18-APT73.248 (SEQ ID NO: 97).

62. The fusion protein of claim 60, wherein the polypeptide having Geranyl diphosphate synthase activity comprises a polypeptide of SEQ ID NO: 49 (GPS1.1), or a functional fragment or functional variant thereof.

63. The fusion protein of claim 60, wherein the fusion protein further comprises a linker polypeptide between the polypeptide having Geranyl diphosphate synthase activity and the polypeptide having prenyltransferase activity, optionally, wherein the linker comprises a polypeptide selected from the amino acid sequence of SEQ ID NO: 50 or SEQ ID NO: 51.

64. A cell comprising the fusion protein of claim 60, wherein the cell:
i) is capable of producing CBGA in the presence of OA and GPP;
ii) is capable of producing CBGVA in the presence of DVA and a GPP;
iii) is capable of making a cannabinoid in the presence of a carbon source and, optionally, hexanoic or butyric acid;
iv) expresses an exogenous membrane transporter that improves OA or DVA uptake, optionally, wherein one or more genes in the cell encoding a protein that exports OA or DVA is down-regulated or deactivated; and/or
v) encodes an exogenous hexanoyl-CoA synthetase and/or butyryl-CoA synthetase.

65. The cell of claim 64, wherein the cell is a yeast cell or a bacterial cell, optionally, wherein the yeast cell is a Yarrowia strain or a Saccharomyces strain.

66. A method of producing CBGA, CBGVA or derivatives thereof comprising culturing the cell of claim 64 under suitable conditions to produce CBGA, CBGVA or derivatives thereof, optionally, wherein the suitable condition comprises supplementing a culture media in which the cell is cultured with at least one of butyric acid, valeric acid, isovaleric acid, hexanoic acid, hexanol, butanol, oleic acid, glycerol or glucose.


SEQUENCE LISTING (105 sequences)

SEQ ID NO: 1 - protein, 286 aa, synthetic construct
MDEVYAAVEQTSRLLDVPCSPDRFEPVWKAFGDQLPDSVLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDG

SEQ ID NO: 2 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDG

SEQ ID NO: 3 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAALYQKGRRCLDG

SEQ ID NO: 4 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGPREFVSGVALAPSGASYYKLAALYQKGRRCLDG

SEQ ID NO: 5 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGVREFVSGVALAPSGASYYKLAALYQKGRRCLDG

SEQ ID NO: 6 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGSREFVSGVALAPSGASYYKLAALYQKGRRCLDG

SEQ ID NO: 7 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRCLDG

SEQ ID NO: 8 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRCLDG

SEQ ID NO: 9 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRKCLDG

SEQ ID NO: 10 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRELDG

SEQ ID NO: 11 - protein, 286 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRPLDG

SEQ ID NO: 12 - protein, 289 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDGRYG

SEQ ID NO: 13 - protein, 289 aa, synthetic construct
MDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDGHRR

SEQ ID NO: 14 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGPREFVSGVALAPSGASYYKLAAIYQKGRRCLDG

SEQ ID NO: 15 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGPREFVSGVALAPSGASYYKLAAIYQKGRRPLDG

SEQ ID NO: 16 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGVREFVSGVALAPSGASYYKLAAIYQKGRRCLDG

SEQ ID NO: 17 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGVREFVSGVALAPSGASYYKLAAIYQKGRRPLDG

SEQ ID NO: 18 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRPLDG

SEQ ID NO: 19 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGPREFVSGVALAPSGASYYKLAAIYQKGRRPLDG

SEQ ID NO: 20 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGVREFVSGVALAPSGASYYKLAAIYQKGRRPLDG

SEQ ID NO: 21 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRCLDG

SEQ ID NO: 22 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEPGREFVSGVALAPSGASYYKLAAIYQKGRRCLDG

SEQ ID NO: 23 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEVGREFVSGVALAPSGASYYKLAAIYQKGRRCLDG

SEQ ID NO: 24 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRPLDG

SEQ ID NO: 25 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRCLDG

SEQ ID NO: 26 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEPGREFVSGVALAPSGASYYKLAAIYQKGRRCLDG

SEQ ID NO: 27 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEPGREFVSGVALAPSGASYYKLAAIYQKGRRPLDG

SEQ ID NO: 28 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEVGREFVSGVALAPSGASYYKLAAIYQKGRRCLDG

SEQ ID NO: 29 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEVGREFVSGVALAPSGASYYKLAAIYQKGRRPLDG

SEQ ID NO: 30 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRPLDG

SEQ ID NO: 31 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEPGREFVSGVALAPSGASYYKLAAIYQKGRRPLDG

SEQ ID NO: 32 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEVGREFVSGVALAPSGASYYKLAAIYQKGRRPLDG

SEQ ID NO: 33 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRCLDG

SEQ ID NO: 34 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEPGREFVSGVALAPSGASYYKLAAIYQKGRRCLDG

SEQ ID NO: 35 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEVGREFVSGVALAPSGASYYKLAAIYQKGRRCLDG

SEQ ID NO: 36 - protein, 286 aa, synthetic construct
MDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRPLDG

SEQ ID NO: 37 - protein, 297 aa, synthetic construct
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSAIGVNYRKNTLNVYLAASAVDTGDKLALLRAFGYPEPDARVRQFIERSFSLYPTFNWDSSAAERICFSVKTQQPGELPAPHDEPTEAFARQVPHVYEGGREFVSAVALAPSGASYYKLAAYYQKARGASNAAFAAKREDAAA

SEQ ID NO: 38 - protein, 286 aa, synthetic construct
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLDG

SEQ ID NO: 39 - protein, 309 aa, synthetic construct
MSDNSIATKILNFGHTCWKLQRPYAVKGMISIACGLFGRELFNNRHLFSWGLMWKAFFALVPILSFNFFAAVMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 40 - protein, 309 aa, synthetic construct
MSDNSIATKILNFGHTCWKLQRPYAVKGIISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 41 - protein, 309 aa, synthetic construct
MSDNSIATKILNFGHTCWKLQRPYAVKGVISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 42 - protein, 309 aa, synthetic construct
MSDNSIATKILNFGHTCWKLQRPYAVKGMISIACGLFGRELFNNRHLFSWGLMWKAFFALVPILSFNFFAACMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 43 - protein, 309 aa, synthetic construct
MSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFMGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 44 - protein, 309 aa, synthetic construct
MSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQLFKSNIMILSHAILAFCLIFQTRELALANYASAPSRQFFEFIWLLYYAEYFVYVFI

SEQ ID NO: 45 - protein, 309 aa, synthetic construct
MSDNSIATKILNFGHTCWKLQRPYAVKGMISIACGLFGRELFNNRHLFSWGLMWKAFFALVPILSFNFFAAAMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 46 - protein, 309 aa, synthetic construct
MSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 47 - protein, 333 aa, synthetic construct
MPATRTPIHPEAAAYKNPRYQSGPLSVIPKSFVPYCELMRLELPHGNFLGYFPHLVGLLYGSSASPARLPANDVVFQAALYIGWTFFMRGAGCAWNDVVDQDFDRKTTRCRVRPVARGAVSTTGANIFALAMVSLAFACISPLPAECQRLGLITTVLSIIYPFCKRVTNFAQVILGMTLAINFILAAYGAGLPAVEAPYTLPTICVTTAITLLVVFYDVVYARQDTADDLKSGVKGMAVLFRNYVEILLTSITLVIAGLLATTGVLVDNGPYFFVFSVAGLLAALLAMIGGIRYRIFHTWNSYSGWFYALAIFNLLGGYLIEYLDQVPMLNKA

SEQ ID NO: 48 - protein, 329 aa, synthetic construct
MSAKVASIPYTNPRYESGPLSAIPKSWVPYFELMRFELPHGYYLGYFPHLVGIMYGASAGPERLPASQLLFQALLYVGWTFCMRGAGCAWNDNIDQDFDRKTERCRTRPIARGAVSTTAGHIFAISFVAAAFLCLAPLPTECHQLGVIVTVLSIIYPFCKRFTNFAQVILGFTLAANFILAAYGAGLPALEQPYTRPTACVTLAITLLVVFYDVVYARQDTADDLKSGVKGMAVLFRNHIEILLAGLTCTIGGLLAVTGIYVENGPYYFVFSVAGLTVALLAMIGGIRYRVFHSWNSYSGWFYVIAIINLMSGYFIEYLGNAPLLARAS

SEQ ID NO: 49 - protein, 344 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 50 - protein, 39 aa, synthetic construct
GGAEAAAKEAAAKAGGSGGGSGGGGSGGSGGGGSGGGGS

SEQ ID NO: 51 - protein, 48 aa, synthetic construct
GGGGSLEDPAVWEAGKVVAKGVGTADITATTSNGLIASSEEADNAATS

SEQ ID NO: 52 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDG

SEQ ID NO: 53 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAALYQKGRRCLDG

SEQ ID NO: 54 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGPREFVSGVALAPSGASYYKLAALYQKGRRCLDG

SEQ ID NO: 55 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGVREFVSGVALAPSGASYYKLAALYQKGRRCLDG

SEQ ID NO: 56 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGSREFVSGVALAPSGASYYKLAALYQKGRRCLDG

SEQ ID NO: 57 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRCLDG

SEQ ID NO: 58 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRCLDG

SEQ ID NO: 59 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRKCLDG

SEQ ID NO: 60 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRELDG

SEQ ID NO: 61 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRPLDG

SEQ ID NO: 62 - protein, 681 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDGRYG

SEQ ID NO: 63 - protein, 681 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDGHRR

SEQ ID NO: 64 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLCFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLDG

SEQ ID NO: 65 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGPREFVSGVALAPSGASYYKLAAIYQKGRRCLDG

SEQ ID NO: 66 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGPREFVSGVALAPSGASYYKLAAIYQKGRRPLDG

SEQ ID NO: 67 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGVREFVSGVALAPSGASYYKLAAIYQKGRRCLDG

SEQ ID NO: 68 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGVREFVSGVALAPSGASYYKLAAIYQKGRRPLDG

SEQ ID NO: 69 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRPLDG

SEQ ID NO: 70 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGPREFVSGVALAPSGASYYKLAAIYQKGRRPLDG

SEQ ID NO: 71 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGVREFVSGVALAPSGASYYKLAAIYQKGRRPLDG

SEQ ID NO: 72 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRCLDG

SEQ ID NO: 73 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEPGREFVSGVALAPSGASYYKLAAIYQKGRRCLDG

SEQ ID NO: 74 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEVGREFVSGVALAPSGASYYKLAAIYQKGRRCLDG

SEQ ID NO: 75 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRPLDG

SEQ ID NO: 76 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRCLDG

SEQ ID NO: 77 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEPGREFVSGVALAPSGASYYKLAAIYQKGRRCLDG

SEQ ID NO: 78 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEPGREFVSGVALAPSGASYYKLAAIYQKGRRPLDG

SEQ ID NO: 79 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEVGREFVSGVALAPSGASYYKLAAIYQKGRRCLDG

SEQ ID NO: 80 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEVGREFVSGVALAPSGASYYKLAAIYQKGRRPLDG

SEQ ID NO: 81 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRPLDG

SEQ ID NO: 82 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEPGREFVSGVALAPSGASYYKLAAIYQKGRRPLDG

SEQ ID NO: 83 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEVGREFVSGVALAPSGASYYKLAAIYQKGRRPLDG

SEQ ID NO: 84 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGGREFVSGVALAPSGASYYKLAAIYQKGRRCLDG

SEQ ID NO: 85 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEPGREFVSGVALAPSGASYYKLAAIYQKGRRCLDG

SEQ ID NO: 86 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEVGREFVSGVALAPSGASYYKLAAIYQKGRRCLDG

SEQ ID NO: 87 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSVLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAAIYQKGRRPLDG

SEQ ID NO: 88 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 89 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGIISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 90 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGVISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 91 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAACMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 92 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAVMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 93 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFMGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 94 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQLFKSNIMILSHAILAFCLIFQTRELALANYASAPSRQFFEFIWLLYYAEYFVYVFI

SEQ ID NO: 95 - protein, 691 aa, synthetic construct
SKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAAMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 96 - protein, 286 aa, synthetic construct
MDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGPREFVSGVALAPSGASYYKLAAIYQKGRRPLDA

SEQ ID NO: 97 - protein, 678 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLLFSMAAGEAHRGELDFDFSLRPEGADPYTTALEHGFIEPTDHPVGSVLAEVGKRFAIASYGVEYGVVGGFKKSYAFFPLDDFPPLAEFARIPSVPPCLAGHVETLTRLGFDDKVSIIGVNYQKKTLNVYLAASAVDTGDKLALLRAFGYPEPDARVRQFIERSFRLYPTFNWDSSAAERICFAVHTNQPGELPAPHDEPTEAFARQVPHVYEGPREFVSGVALAPSGASYYKLAAIYQKGRRPLDA

SEQ ID NO: 98 - protein, 329 aa, synthetic construct
MSAKVASIPYTNPRYESGPLSAIPKSWVPYFELMRFELPHGYYLGYFPHLVGIMYGASAGPERLPASQLLFQALLYVGWTFCMRGAGCAWNDNIDQDFDRKTERCRTRPIARGAVSTTAGHIFAISFVAAAFLCLAPLPTECHQLGVIFTVLSIIYPFCKRFTNFAQVILGFTLAANFILAAYGAGLPALEQPYTRPTACVTLAITLLVVFYDVVYARQDTADDLKSGVKGMAVLFRNHIEILLAGLTCTIGGLLAVTGIYVENGPYYFVFSVAGLTVALLAMIGGIRYRVFHSWNSYSGWFYVIAIINLMSGYFIEYLGNAPLLARAS

SEQ ID NO: 99 - protein, 329 aa, synthetic construct
MSAKVASIPYTNPRYESGPLSAIPKSWVPYFELMRFELPHGYYLGYFPHLVGIMYGASAGPERLPASQLLFQALLYVGWTFCMRGAGCAWNDNIDQDFDRKTERCRTRPIARGAVSTTAGHIFAISFVAAAFLCLAPLPTECHQLGVIHTVLSIIYPFCKRFTNFAQVILGFTLAANFILAAYGAGLPALEQPYTRPTACVTLAITLLVVFYDVVYARQDTADDLKSGVKGMAVLFRNHIEILLAGLTCTIGGLLAVTGIYVENGPYYFVFSVAGLTVALLAMIGGIRYRVFHSWNSYSGWFYVIAIINLMSGYFIEYLGNAPLLARAS

SEQ ID NO: 100 - protein, 329 aa, synthetic construct
MSAKVASIPYTNPRYERGPLSAIPKSWVPYFELMRFELPHGYYLGYFPHLVGIMYGASAGPERLPASQLLFQALLYVGWTFCMRGAGCAWNDNIDQDFDRKTERCRTRPIARGAVSTTAGHIFAISFVAAAFLCLAPLPTECHQLGVIVTVLSIIYPFCKRFTNFAQVILGFTLAANFILAAYGAGLPALEQPYTRPTACVTLAITLLVVFYDVVYARQDTADDLKSGVKGMAVLFRNHIEILLAGLTCTIGGLLAVTGIYVENGPYYFVFSVAGLTVALLAMIGGIRYRVFHSWNSYSGWFYVIAIINLMSGYFIEYLGNAPLLARAS

SEQ ID NO: 101 - protein, 329 aa, synthetic construct
MSAKVASIPYTNPRYESGPLSAIPKSWVPYFELMRFELPHGYYLGYFPHLVGIMYGASAGPERLPASQLLFQALLYVGWTFCMRGAGCAWNDNIDQDFDRKTERCRTRPIARGAVSTTAGHIFAISFVAAAFLCLAPLPTECHQLGVIVTVLSIIYPFCKRFTNFAQVILGFTLAANFILAAYGAGLPALEQPYTRPTACVTLAITLLVVFYDVVYARQDTADDLKSGVKGMAVLFRNHIEILLAGLTCTIGGLLAVTGIYVENGPYYFVFSVAGLTVALLAMIGGIRYRVFHSWNSYPGWFYVIAIINLMSGYFIEYLGNAPLLARAS

SEQ ID NO: 102 - protein, 329 aa, synthetic construct; identical to SEQ ID NO: 101

SEQ ID NO: 103 - protein, 329 aa, synthetic construct
MSAKVASIPYTNPRYESGPLSAIPKSWVPYFELMRFELPHGYYLGYFPHLVGIMYGASAGPERLPASQLLFQALLYVGWTFCMRGAGCAWNDNIDQDFDRKTERCRTRPIARGAVSTTAGHIFAISFVAAAFLCLAPLPTECHQLGVIVTVLSIIYPFCKRFTNFAQVILGFTLAANFILAAYGAGLPALEQPYTRPTACVTLAITLLVVFYDVVYARQDTADDLKSGVKGMAVLFRNHIEILLAGLTCTIGGLLAVTGIYVENGPYYFVFSVAGLTVALLAMIGGIRYRVFHSWNSYWGWFYVIAIINLMSGYFIEYLGNAPLLARAS

SEQ ID NO: 104 - protein, 329 aa, synthetic construct
MSAKVASIPYTNPRYESGPLSAIPKSWVPYFELMRFELPHGYYIGYFPHLVGIMYGASAGPERLPASQLLFQALLYVGWTFCMRGAGCAWNDNIDQDFDRKTERCRTRPIARGAVSTTAGHIFAISFVAAAFLCLAPLPTECHQLGVIVTVLSIIYPFCKRFTNFAQVILGFTLAANFILAAYGAGLPALEQPYTRPTACVTLAITLLVVFYDVVYARQDTADDLKSGVKGMAVLFRNHIEILLAGLTCTIGGLLAVTGIYVENGPYYFVFSVAGLTVALLAMIGGIRYRVFHSWNSYSGWFYVIAIINLMSGYFIEYLGNAPLLARAS

SEQ ID NO: 105 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFLSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK